(19) United States
(12) Patent Application Publication
(10) Pub. No.: US 2021/0137830 A1
(43) Pub. Date: May 13, 2021

(54) TOPICAL DETOMIDINE FORMULATIONS

(71) Applicant: CLEXIO BIOSCIENCES LTD., Jerusalem (IL)

(72) Inventors: Mark ZAMANSKY, Givat Shaul (IL); Erez KOREN, Givat Shaul (IL)

(21) Appl. No.: 17/259,732

(22) PCT Filed: Jul. 11, 2019
(86) PCT No.: PCT/IB2019/055935

§ 371 Date: Jan. 12, 2021

Related U.S. Application Data
(60) Provisional application No. 62/696,473, filed on Jul. 11, 2018.

(51) Int. Cl. A61K 9/00; A61K 31/4174; A61K 45/06; A61K 47/14; A61K 9/06; A61K 9/107; A61K 9/12; A61K 47/32; A61K 47/34; A61K 9/16; A61K 9/51; A61K 9/70; A61K 47/44

(52) U.S. Cl. CPC A61K 9/0014; A61K 31/4174; A61K 45/06; A61K 47/14; A61K 9/06; A61K 9/1075; A61K 47/44; A61K 47/32; A61K 47/34; A61K 9/1647; A61K 9/5153; A61K 9/7015; A61K 9/122

(57) ABSTRACT

Disclosed herein are topical formulations comprising about 0.001 to about 3 wt % detomidine or a salt thereof; and, a carrier that is suitable for topical administration to a subjects skin, wherein the carrier comprises hydrophilic and hydrophobic phase members in specified ratios, and wherein the detomidine is fully dissolved and chemically stable in the hydrophilic phase member. The topical formulations may be provided in the form of emulsions, creams, ointments, gels, spray patches, dispersions, and other specified forms. Also provided are methods for providing prolonged, non-systemic treatment for pain in a subject in need thereof comprising topically administering to the subject the presently disclosed topical formulations.

### CROSS-REFERENCE TO RELATED APPLICATIONS

This application claims the benefit of priority to U.S. Provisional App. No. 62/696,473, filed Jul. 11, 2019, the entire contents of which are incorporated herein by reference.

### TECHNICAL FIELD

The present disclosure pertains to formulations that contain detomidine and methods of treating pain using such formulations.

### BACKGROUND

Pain is the most common symptom accompanying, to some degree, almost every medical condition that humans experience. When it is severe enough, it interferes with a patient's ability to function and with quality of life. A common form of pain is that which is associated with peripheral neuropathy, a condition that develops as a result of damage to the peripheral nervous system.

Symptoms of peripheral neuropathy can range from numbness or tingling, to pricking sensations (paresthesia), or muscle weakness. Areas of the body may become abnormally sensitive leading to an exaggeratedly intense or distorted experience of touch (allodynia). When symptoms are severe, they may include burning pain, muscle wasting, paralysis, or organ or gland dysfunction.

There are numerous forms of peripheral neuropathy, and such forms can follow different patterns. Symptoms can be acute or chronic, and can occur over a period of days, weeks, or years. Acute neuropathies, which include Guillain-Barré syndrome, include symptoms that appear suddenly, progress rapidly, and resolve slowly as damaged nerves heal. Symptoms associated with chronic neuropathies can include a subtle start and slow progression, and periods of relief followed by relapse can occur. Other individuals may experience symptoms that reach a certain level of severity and then stay the same for extended periods of time, and still other chronic neuropathies worsen over time.

Diabetic neuropathy represents one of the most common forms of peripheral neuropathy. In this form, nerve damage is progressive: pain and numbness can first occur in both feet, followed by a gradual progression up both legs. Later, the fingers, hands, and arms may become affected. Another form of peripheral neuropathy is postherpetic neuralgia, which is a complication of shingles, which is in turn caused by the chickenpox (herpes zoster) virus. Postherpetic neuralgia affects nerve fibers and skin and can cause burning pain that persists for long periods following disappearance of the rash and blisters of shingles.

Although it is generally difficult to control, various strategies have been deployed for treating neuropathic pain. Over-the-counter analgesics such as nonsteroidal anti-inflammatory drugs (NSAIDs) can be used to treat mild pain. Medications that are used for chronic neuropathic pain fall under several classes of drugs: antidepressants, anticonvulsant medications, antiarrythmic medications, and narcotic agents. Antidepressants (such as tricyclic antidepressants such as amitriptyline or newer serotonin-norepinephrine reuptake inhibitors such as duloxetine hydrochloride or venlafaxine) and anticonvulsant medications (such as gabapentin, pregabalin, topiramate, and carbamazepine, although other medications used for treating epilepsy) presently represent the most effective types of medications for controlling neuropathic pain. Mexiletine is an antiarrythmic medication that may also be used for treatment of chronic painful neuropathies. Narcotic agents have been prescribed for pain that does not respond to any of the preceding medications. However, narcotic medications can lead to dependence and addiction, and there use is therefore limited to situations in which other treatments have failed.

Certain topically administered medications have been used for addressing neuropathic pain. Lidocaine, an anesthetic agent, and capsaicin, which modifies peripheral pain receptors, represent two prominent examples. Topical agents are generally most appropriate for localized chronic pain, such as that deriving from herpes zoster neuralgia (shingles).

A need persists for additional strategies for alleviating pain associated with a peripheral neuropathy and other conditions imposing acute or chronic effects.

### SUMMARY

The present disclosure provides topical formulations comprising about 0.001 to 3% by weight of detomidine or a salt thereof, and, a carrier that is suitable for topical administration to a human subject's skin, wherein the carrier comprises a hydrophilic phase member and a hydrophobic phase member in a ratio of not more than 49.9%, and, wherein the detomidine is fully dissolved and chemically stable in the hydrophilic phase member.

Also disclosed are topical formulations comprising about 0.001 to 10% by weight of detomidine or a salt thereof, and, a carrier that is suitable for topical administration to a human subject's skin, wherein the carrier comprises a hydrophobic phase member and a hydrophilic phase member in a ratio of not more than 10%, and, wherein the detomidine is fully dissolved and chemically stable in the hydrophilic phase member.

The present disclosure also provides topical formulations comprising an oil-in-water emulsion in which porous microparticles or nanoparticles are dispersed, at least some of the pores of said microparticles or nanoparticles being loaded with detomidine or a salt thereof, a further active pharmaceutical agent, or both.

Also provided are sprayable compositions comprising detomidine or a salt thereof that forms a transdermal patch when sprayed onto the subject's skin.

The present disclosure also pertains to methods for providing prolonged, non-systemic treatment for pain in a subject in need thereof comprising topically administering to the subject a formulation according to any of the preceding types.

Also disclosed herein are methods for providing treatment for rosacea in a human subject in need thereof comprising topically administering to the human subject a topical formulation according to any one of the embodiments disclosed herein.

Additional embodiments will be readily apparent on the basis of the disclosure herein.

### BRIEF DESCRIPTION OF THE DRAWINGS

FIG. 1 depicts rheology plots for high internal phase oil-in-water emulsion-based formulations according to the present disclosure at 1 day and 6 weeks.

FIG. 2 depicts rheology plots for additional high internal phase oil-in-water emulsion-based formulations according to the present disclosure at 1 day and 6 weeks.

### DETAILED DESCRIPTION OF ILLUSTRATIVE EMBODIMENTS

The present inventions may be understood more readily by reference to the following detailed description taken in connection with the accompanying figures and examples, which form a part of this disclosure. It is to be understood that these inventions are not limited to the specific products, methods, conditions or parameters described and/or shown herein, and that the terminology used herein is for the purpose of describing particular embodiments by way of example only and is not intended to be limiting of the claimed inventions.

The entire disclosures of each patent, patent application, and publication cited or described in this document are hereby incorporated herein by reference.

As employed above and throughout the disclosure, the following terms and abbreviations, unless otherwise indicated, shall be understood to have the following meanings.

In the present disclosure the singular forms “a,” “an,” and “the” include the plural reference, and reference to a particular numerical value includes at least that particular value, unless the context clearly indicates otherwise. Thus, for example, a reference to “a particle” is a reference to one or more of such particles and equivalents thereof known to those skilled in the art, and so forth. Furthermore, when indicating that a certain element “may be” X, Y, or Z, it is not intended by such usage to exclude in all instances other choices for the element.

When values are expressed as approximations, by use of the antecedent “about,” it will be understood that the particular value forms another embodiment. As used herein, “about X” (where X is a numerical value) preferably refers to ±10% of the recited value, inclusive. For example, the phrase “about 8” preferably refers to a value of 7.2 to 8.8, inclusive; as another example, the phrase “about 8%” preferably refers to a value of 7.2% to 8.8%, inclusive. Where present, all ranges are inclusive and combinable. For example, when a range of “1 to 5” is recited, the recited range should be construed as optionally including ranges “1 to 4”, “1 to 3”, “1-2”, “1-2 & 4-5”, “1-3 & 5”, and the like. In addition, when a list of alternatives is positively provided, such a listing can also include embodiments where any of the alternatives may be excluded. For example, when a range of “1 to 5” is described, such a description can support situations whereby any of 1, 2, 3, 4, or 5 are excluded; thus, a recitation of “1 to 5” may support “1 and 3-5, but not 2”, or simply “wherein 2 is not included.” The phrase “at least about x” is intended to embrace both “about x” and “at least x”.

The present disclosure relates, inter alia, to topical formulations comprising about 0.001 to about 10 wt % detomidine or a salt thereof, and methods for treating pain by topical administration of such formulations. As noted above, pain is perhaps the most common symptom accompanying nearly every medical condition that a human can experience, and certain forms of pain, including those deriving from peripheral neuropathy, remain difficult to treat. The present inventors have developed new topical formulations containing detomidine or a salt thereof that provide relief from pain over an extended period of time. Although the present formulations can provide treatment of numerous forms of localized pain, of particular benefit is the ability of the formulations to treat pain deriving from a peripheral neuropathy. These and other advantages that are conferred by the present formulations and methods using such formulations are described more fully herein.

Detomidine is a synthetic alpha-2 adrenoreceptor agonist with sedative and analgesic properties. It is presently sold by prescription under the trade name DORMOSEDAN® (Zoetis Services LLC, Parsippany, N.J.) as a sedative and anesthetic premedication in connection with a variety of minor surgical and diagnostic procedures on horses and other large animals. It is commonly combined with butorphanol in order to increase the degree of analgesia and depth of sedation, and may also be used with ketamine for intravenous anesthesia of short duration. The route of administration of DORMOSEDAN® injection is typically intramuscular or intravenous, but the drug is also available as a gel (DORMOSEDAN GEL®) that may be administered by the sublingual route.

### Predominantly Hydrophobic Topical Formulations

As described above, provided are topical formulations comprising about 0.001 to 3% by weight of detomidine or a salt thereof; and, a carrier that is suitable for topical administration to a human subject's skin, wherein the carrier comprises a hydrophilic phase member and a hydrophobic phase member in a ratio of not more than 49.9%, and wherein the detomidine is fully dissolved and chemically stable in the hydrophilic phase member.

The formulations contain about 0.001 to about 3 wt % detomidine or a salt thereof. For example, the formulations may include about 0.005 to about 2, about 0.005 to about 3, about 0.003 to about 3, about 0.008 to 3, about 0.01 to about 3, about 0.01 to about 2, about 0.01 to about 1.5, about 0.01 to about 1, about 0.01 to about 2, 0.033 to about 1, 0.033 to about 0.33, about 0.08 to about 0.5, about 0.08 to about 0.2, about 0.05 to about 3, about 0.05 to about 2, about 0.05 to about 1, about 0.1 to about 3, about 0.1 to about 2.5, about 0.1 to about 2, about 0.1 to about 1.5, about 0.1 to about 1, about 0.33 to about 1, about 0.5 to about 1, or about 0.5 to about 0.75 wt % detomidine or salt thereof, or about 0.001, about 0.002, about 0.003, about 0.005, about 0.007, about 0.008, about 0.009, about 0.01, about 0.03, about 0.05, about 0.075, about 0.08, about 0.09, about 0.1, about 0.2, about 0.3, about 0.33, about 0.5, about 0.7, about 0.75, about 0.8, about 1.0, about 1.2, about 1.25, about 1.33, about 1.4, about 1.5, about 1.6, about 1.7, about 1.8, about 1.9, about 2.0, about 2.1, about 2.2, about 2.3, about 2.33, about 2.4, about 2.5, about 2.6, about 2.7, about 2.8, about 2.9, or about 3 wt % of detomidine or salt thereof.

The detomidine may be present in the formulations in the free base form or as a salt. Unless specified otherwise, reference to “detomidine” in the present disclosure can refer to detomidine in a free base form, or to a salt of detomidine. Those of ordinary skill in the art can readily identify exemplary pharmaceutically acceptable salt forms of detomidine. Suitable pharmaceutically acceptable salts of detomidine include detomidine bitartrate, detomidine bitartrate hydrate, detomidine hydrochloride, detomidine p-toluenesulfonate, detomidine phosphate, detomidine thiosemicarbazone, detomidine sulfate, detomidine trifluoroacetate, detomidine hemipentahydrate, detomidine bitartrate hemipentahydrate, detomidine pentafluoropropionate, detomidine p-nitrophenylhydrazone, detomidine o-methyloxime, detomidine semicarbazone, detomidine hydrobromide, detomidine mucate, detomidine oleate, detomidine phosphate dibasic, detomidine phosphate monobasic, detomidine inorganic salt, detomidine organic salt, detomidine acetate trihydrate, detomidine bis(heptafluorobutyrate), detomidine bis(methylcarbamate), detomidine bis(pentafluoropropionate), detomidine bis(pyridine carboxylate), detomidine bis(trifluoroacetate), detomidine chlorhydrate, and detomidine sulfate pentahydrate. In certain embodiments of the presently disclosed dosage forms, the detomidine is present as the hydrochloride salt.

The formulations also include a carrier that is suitable for topical administration to a subject's skin. In addition to the hydrophilic phase member and hydrophobic phase member, which are described more fully below, the carrier may include, for example, a solubilizer, a buffer, or both. As described below, the formulation may also include one or more additional components in order to produce the topical form, such as thickening or gelling agents, preservatives, antioxidants, permeation enhancers, emulsifying agents, emollients, or humectants.

In the present formulations, the detomidine or salt thereof is fully dissolved in the hydrophilic phase member. In other words, the present formulations do not include any precipitated or crystalline detomidine or salt thereof. The presently disclosed formulations are also chemically stable with respect to the dissolution state of the detomidine or salt thereof. As used herein, “chemically stable” means that there is no or nearly no precipitation or crystallization of the detomidine or salt thereof (for example, under visual examination, after magnified examination under polarized light, or both) when the formulation is stored under standard conditions, e.g., when stored at a standard storage temperature of 15-25° C., over a desired storage period, such as for at least 12 hours, 24 hours, 2 days, 3 days, 4 days, 5 days, 6 days, one week, 9 days 10 days, 12 days, two weeks, 18 days, three weeks, one month, two months, three months, six months, eight months, 10 months, one year, 14 months, 16 months, 18 months, 20 months, 22 months, or two years.

The concentration of detomidine or salt thereof that is dissolved in the hydrophilic phase member is up to 40% w/w. For example, the concentration of detomidine or salt thereof that is dissolved in the hydrophilic phase member may be about 0.01-38, 0.04-35, 0.1-30, 0.5-30, 1-30, 3-30, 5-30, 7-30, 10-30, 5-25, 7-25, 8-25, 10-25, 10-22, 10-20, 12-20, or 15-20% w/w, or may be about 0.01, 0.05, 0.1, 0.3, 0.5, 1.0, 1.5, 2, 4, 6, 8, 10, 12, 15, 17, 20, 22, 25, 27, 30, 32, 35, 37, or 40% w/w.

The hydrophilic phase member may represent any predominantly hydrophilic material in which detomidine or a salt thereof can be dissolved at any of the concentrations described above. Exemplary hydrophilic phase members include water, glycerol, polypropylene glycol, polyethylene glycol, ethanol, benzyl alcohol, 1,3-propanediol, 1,2-pentanediol, propylene carbonate, 2-(2-ethoxyethoxy)ethanol, dimethyl isosorbide, tetraglycol, pyrrolidone, dimethylacetamide, caprylocaproyl polyoxyl-8 glycerides, hexylene glycol, butylene glycol, or any combination thereof.

The hydrophilic phase member may also comprise an aqueous buffer solution. For example, the hydrophilic phase member may comprise 0.01 to 1.0M citrate, phosphate, Tris, carbonate, succinate, tartrate, borate, imidazole, maleate, or phthalate buffer at pH 4.5-9.0. For example, the buffer may be effective to maintain the pH of the formulations at about 5.0 to about 9, about 5.0 to about 9, about 5.0 to about 8.5, about 5.0 to about 8.2, about 5.0 to about 8, about 5.0 to about 6.0, about 5.0 to about 5.5, about 5.2 to about 9, about 5.2 to about 8.5, about 5.2 to about 8.2, about 5.2 to about 8, about 5.2 to about 7, about 5.2 to about 6, about 5.2 to about 5.5, about 5.5 to about 9, about 5.5 to about 8.5, about 5.5 to about 8.2, or about 5.5 to about 8, or at about 4.5, about 4.6, about 4.7, about 4.8, about 4.9, about 5.0, about 5.1, about 5.2, about 5.3, about 5.4, about 5.5 about 5.6, about 5.7, about 5.8, about 5.9, about 6.0, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5, about 7.6, about 7.7, about 7.8. about 7.9, about 8.0, about 8.1, about 8.2, about 8.3, about 8.4, about 8.5, about 8.6, about 8.7, about 8.8, about 8.9, or about 9.0. In certain embodiments, the pH of the formulation may be about 4.5 to about 7, for example about 4.5 to about 6, about 5 to about 6, or about 5.5. In certain embodiments, the pH of the formulation may be about 7 or lower, such as about 6 or lower. Buffers other than those specifically listed above that may be used to maintain the pH of the formulation at a desired level can be readily identified by those of ordinary skill in the art. Exemplary aqueous buffer solutions for the hydrophilic phase member include 0.1M citrate buffer at pH 6.1, 0.1M phosphate buffer at pH 6.2, 0.1M phosphate buffer at pH 7.2, 0.1M Tris buffer at pH 8.2, 0.4M Tris buffer at pH 8.2, water with 60% w/w PEG 3350 in pH 6.2 buffer, and 0.4M Tris buffer at pH 8.2 with 30% Glycerin and 40% PEG 3350. Other aqueous buffer solutions are disclosed in the examples below.

The hydrophobic phase member may be, for example, an aromatic hydrocarbon, an alkane, a cycloalkane, an alkyne, a terpene, an organic oil, a mineral oil, or any combination thereof. Exemplary hydrophilic phase members include mineral oil, isopropyl isostearate, isostearyl isostearate, alkyl benzoate, butyl stearate, diisopropyl adipate, diethylhexyl adipate, caprilic/capric triglyceride, isocetyl stearate, isopropyl myristate, isopropyl palmitate, lauryl lactate, myristil myristate, ethylhexyl cocoate, ethylhexyl palmiatate, ethylhexyl pelagronate, ethylhexyl stearate, diethylhexyl succinate, propylene glycol dicaprylate/dicaprate, PPG-2 myristyl ether propionate, pentaerythrityl tetracaprylate/caprate, pentaerythrityl tetraisostearate, PEG 2 stearyl ether, steareth-21, and isotridecyl isononanoate. An exemplary genus of hydrophobic phase members is medium chain triglycerides. Further hydrophobic phase members that represent fatty acid esters are disclosed in U.S. Pub. No. 2012/0201863, the entire contents of which are incorporated herein by reference.

As described above, the hydrophilic phase member and a hydrophobic phase member are present in the carrier in a ratio not more than about 49.9%. In other words, the hydrophobic phase member predominates within the carrier. For example, the hydrophilic phase member and the hydrophobic phase member are present in the carrier in a ratio of less than about 49, 47, 45, 40, 35, 30, 25, 20, 15, 12, 10, 8, 5, 3, or 2%, or the hydrophilic phase member and the hydrophobic phase member may be present in the carrier in a ratio of about 49, 47, 45, 42, 40, 37, 35, 32, 30, 27, 25, 22, 20, 18, 17, 16, 15, 12, 10, 8, 5, or 2%. In a particular embodiment, the hydrophilic phase member and the hydrophobic phase member are present in the carrier in a ratio not more than about 20%.

In certain embodiments, the present formulations may be prepared by (i) dissolving the detomidine or salt thereof in the hydrophilic phase member, and (ii) combining the hydrophilic phase member containing the dissolved detomidine or salt thereof with the hydrophobic phase member in order to form the topical formulation. Additional details regarding these and other processes for preparing topical formulations of detomidine or a salt thereof are provided in the examples below.

The present formulations may further comprise one or more additional components for enhancing a desired feature relating to the stability, texture, viscosity, storability, or some other characteristic of the formulation. For example, the formulations may further comprise one or more of a thickening agent, a preservative, a permeation enhancer, a wax, an emulsifying agent, an emollient, a humectant, a conditioning agent, antioxidant, and a viscosity regulator.

The present formulations may contain detomidine or a salt thereof as the only therapeutic agent. In other embodiments, the formulations may include a further therapeutic agent in addition to the detomidine or a salt thereof. For example, the formulations may further comprise an analgesic (such as an NSAID, an opioid, or acetaminophen), an antidepressant agent (such as a tricyclic antidepressant), an anticonvulsant agent, or a local anesthetic (such as lidocaine, prilocaine, tetracaine, benzocaine, proxymetacaine, and the like). As another example, the formulations may further comprise one or more additional α-2-adrenergic agonists. Preferred α-2-adrenergic agonists include clonidine, romifidine, brimonidine, dexmedetomidine, and salts thereof.

The present formulations are designed for topical application to a subject's skin. Accordingly, the formulations are not configured for oral administration or for injection. Put differently, the formulations are non-oral and non-injectable.

The formulations can include a volatile solvent that at least partially evaporates from the skin surface following application. For example, in certain embodiments, the buffer component is aqueous, and the water that is contained within the aqueous buffer represents the volatile solvent. The portion of the formulation that remains following at least partial evaporation can be referred to as the “nonvolatile” or “residual” phase, and the volatile element(s) of the formulation that evaporate from the skin surface represents the “volatile” phase. In the present formulations, the detomidine or salt thereof is at or close to its saturation point within the nonvolatile phase following evaporation of the volatile phase. For example, the detomidine or salt thereof may be present in the residual phase following topical application of the instant formulations at or greater than about 75% of the saturation point of the active agent. In some embodiments, the detomidine or salt thereof is present in the residual phase at or greater than about 77, about 80, about 82, about 84, about 85, about 87, about 88, about 90, about 92, about 94, about 95, about 96, about 97, about 98, or about 99% of the saturation point for the detomidine or salt thereof.

In certain embodiments, the formulations may include an inert excipient that assists with increasing the concentration of the detomidine or salt thereof in the residual phase following topical application. In effect, such excipients can cause “salting out” of the detomidine or salt thereof from the other components of the residual phase, which can have the effect of increasing the activity of the detomidine or salt thereof on the surface of the subject's skin and promote permeability of the drug through the skin. Such inert excipients can include, for example, a polyol or simple sugar, such as sucrose, dextrose, trehalose, mannitol, sorbitol, or xylitol.

The present formulations may provide prolonged, substantially non-systemic treatment for pain. The period of time over which the formulations can provide treatment for pain is up to 24 hours when topically applied once a day. In certain embodiments, the formulations are preferably applied twice per day, and in such instances the treatment of pain that is provided by a first of the two topical administrations has a duration that lasts until the second topical administration, and the second daily topical administration has a duration that lasts until the following day's first topical administration. As used herein, “substantially non-systemic” refers to a treatment effect that is localized to the bodily region (for example, body part) to which the formulation is topically applied, with a minimal or no medically relevant effect outside of that bodily region, or simply no minimal or no medically relevant systemic effect.

Being designed for topical application, the present formulations may take any appropriate form, including, for example, that of a cream, foam, gel, lotion, or ointment. As required and as described herein, the formulations according to the present disclosure may further comprise one or more additional components in order to produce the topical form, such as thickening or gelling agents, preservatives, antioxidants, permeation enhancers, emulsifying agents, emollients, or humectants.

Thickening or gelling agents can include, but are not limited to, hydroxymethyl cellulose, hydroxyethyl cellulose, hydroxypropyl cellulose, hydroxypropylmethyl cellulose, xanthan gum, carbomers (acrylates and acrylic acid and its derivatives polymers, such as Carbopol® 980 (crosslinked polyacrylate polymer)), povidones (e.g., polyvinylpyrrolidone), Poloxamers, Polyamide-3 (e.g., OleoCraft™ HP33), and other appropriate agents.

Preservatives can include, but are not limited to, benzalkonium chloride, parabens, sorbic acid and its salts, benzoic acid and its salts, cetrimonium bromide and chloride salts, phenoxyethanol, and other agents.

Antioxidants can include, but are not limited to, sodium metabisulfite, ascorbic acid, tocopheryl acetate (for purely aqueous formulations), and BHT or BHA (for hydrophobic formulations).

Permeation enhancers can include, but are not limited to, Transcutol® P (highly purified diethylene glycol monoethyl ether EP/NF) or dimethylisosorbide (DMI).

Emulsifying agents can include, but are not limited to, Tweens, Spans, poloxamers (124, 407, 188), Brij S2 and Brij 721, Crodex M (cetearyl alcohol and potassium cetyl Phosphate), Crodafos CES (cetearyl alcohol and dicetyl phosphate and Ceteth-10 phosphate (Crodafos CES), Cithrol DPHS (PEG 30 Dipolyhydroxystearate), cyclopentasiloxane, or macrogol hydroxystearate.

Emollients can include, but are not limited to, Migliol 810 or 812 (caprylic-capric triglycerides), Isoporpyl Isostearate (Crodamol IPIS), Isostearyl Isostearate (Crodamol ISIS) or PPG-11 Stearyl Ether (Arlamol PS11E).

Humectants can include, but are not limited to, glycerin, propylene glycol, 1,3-propanediol, or 1,2-pentanediol.

In certain embodiments, examples of which are described more fully infra, the present formulations may comprise a foam. Foams according to the present disclosure may include a hydrophobic phase member that comprises, for example, a medium chain triglyceride, mineral oil, or both. The hydrophilic phase member in the foams may include, for example, one or more of propylene glycol, hexylene glycol, 1,3-propanediol, 1,2-pentanediol or water.

The present formulations may comprise a cream. In cream formulations, the hydrophobic phase member may comprise, for example, mineral oil, isopropyl isostearate, isostearyl isostearate, alkyl benzoate, butyl stearate, diisopropyl adipate, diethylhexyl adipate, caprilic/capric triglyceride, isocetyl stearate, isopropyl myristate, isopropyl palmitate, lauryl lactate, myristil myristate, ethylhexyl cocoate, ethylhexyl palmiatate, ethylhexyl pelagronate, ethylhexyl stearate, diethylhexyl succinate, propylene glycol dicaprylate/dicaprate, PPG-2 myristyl ether propionate, pentaerythrityl tetracaprylate/caprate, pentaerythrityl tetraisostearate, isotridecyl isononanoate, or any combination thereof. The hydrophilic phase member may be, for example, glycerol, propylene glycol, water, 1,3-propanediol, 1,2-pentanediol, hexylene glycol, butylene glycol, or any combination thereof.

Cream formulations may further comprise a fatty alcohol, an ester of a fatty alcohol, or both, an emulsifier, an emollient, or any combination thereof, including each of these components.

Although certain components are disclosed herein separately from the hydrophilic and hydrophobic phase members, it is possible for any of such components effectively to function as an aspect of the hydrophobic or hydrophilic components of the formulations. For example, Labrasol® (caprylocaproyl macrogol-8/polyoxyl-8 glycerides), which is an oil-in-water emulsifier, is also a good solvent for detomidine HCl, and could therefore function as an aspect of the hydrophilic phase member. Thus, emulsifiers that are water soluble, such as Labrasol/Pluronics and other similar polyoxyl and polyglyceryl glycerides, Tweens such as polysorbate 20, 40, 60, 65, 80 and others, Cremophors/Crodurets and other similar Polyoxyl 40, 50, 60 castor oils, liquid Poloxamers including, but not limited to, Poloxamer 124, may effectively have dual functionality (as emulsifiers and effective members of the hydrophilic phase member), but for purposes of the present disclosure, are describe separately from the hydrophilic phase, per se. Likewise, water-in-oil emulsifiers, like Span® or other sorbitan laurate, palmitate, stearate, oleate esters, emuslfiers, Arlacel® and other similar glyceryl esters of laurate, palmitate, stearate, oleate emulsifiers, and the like, as well as ionic surfactants such as sodium lauryl sulfate, cetrimonium bromide, benzalkonium chloride, Crodafos™-type agents, and the like, can effectively be part of the hydrophilic phase. When a component of any of the presently disclosed formulations possesses a dual functionality of the type referred to above (e.g., is an emulsifier, and due to chemical characteristics, is effectively part of the hydrophilic phase), the ratio of the hydrophilic phase member to the hydrophobic phase member will account for the presence of such a component.

The presently disclosed formulations comprising a hydrophilic phase member and a hydrophobic phase member in a ratio of not more than 49.9% may further include porous microparticles or nanoparticles, at least some of the pores of the microparticles or nanoparticles being loaded with detomidine or a salt thereof, a further active pharmaceutical agent, or both. Such formulations therefore represent a dispersion of the microparticles or nanoparticles. The microparticles or nanoparticles may have pores with any appropriate diameter for accepting and releasing the detomidine and any additional active agent. For example, the microparticles or nanoparticles may have an average pore diameter of about 0.05 to 0.5 μm. The microparticles or nanoparticles may be comprise poly(lactic-co-glycolic acid), poly(lactic acid), poly(l-lactic) acid, chitosan, methyl methacrylate/ethylene glycol dimethacrylate crosspolymer, a cellulose ether polymer, or a methacrylate polymer. Exemplary cellulose ether polymers for forming micro particles and nanoparticles include ethyl cellulose, hydroxypropyl methyl cellulose phthalate, hydroxypropyl methyl cellulose acetate succinate, and cellulose acetate. Exemplary methacrylate polymers include Eudragit® polymers (e.g., Eudragit® L100, Eudragit® RL100, Eudragit® RS100, or Eudragit® E). Those skilled in the art can readily identify appropriate methods for forming micro- or nanoparticles using these or other materials.

When present in “soft” particles, such as liposomes, the detomidine or salt thereof will be dissolved. However, in “solid” particles like PLGA or hydrophobic cellulose, the detomidine or salt thereof will be present in a precipitated form. Accordingly, the presence of micro- or nanoparticles having pores in at least some of which detomidine or a salt thereof is loaded will represent a minor exception to the requirement that the present formulations do not contain any undissolved detomidine or salt thereof. For purposes of the present disclosure such formulations (i.e., those containing micro- or nanoparticles having pores in at least some of which detomidine or a salt thereof is loaded) are deemed to be consistent with the general requirement that the detomidine is fully dissolved.

The present formulations that include a hydrophilic phase member and a hydrophobic phase member in a ratio of not more than 49.9% may comprise a microemulsion gel (also referred to as a gelled microemulsion). Exemplary formulations are described infra. Microemulsion gels may include, for example, up to 10% water, up to 20% by weight of a surfactant, a co-surfactant, or both, and 80-100% by weight of an oil. In other instances, a microemulsion gel may include up to 10% by weight water, 30-40% by weight of a surfactant, a co-surfactant, or both, and 60-70% by weight of an oil. In such systems, microemulsions may be gelled using colloidal or mesoporous silica, magnesium stearate, sodium stearyl fumarate, or another gelling agent or emulsifier. When a silicone-based emulsifier or gelling agent, the silicone-based emulsifier may be, for example, liquid alkylmethyl silicone polyether copolymer, ethoxylated and propoxylated silicone surfactant, cyclopentasiloxane, or cyclopentasiloxane with polyethylene glycol/polypropylene glycol-19/19 dimethicone. Nonlimiting examples of oils for use in the present microemulsion gels include oleic acid, Miglyol® 810 or 812 (triglycerides of the fractionated plant fatty acids C₈ and C₁₀), and mineral oil.

The formulations according to the present disclosure that include a hydrophilic phase member and a hydrophobic phase member in a ratio of not more than 49.9% may comprise an oil-in-water emulsion. Such embodiments may take the form of a cream, for example. The hydrophobic phase member in exemplary oil-in-water cream formulations may be hydrophobic phase member comprises PEG 2 stearyl ether, steareth-21, mineral oil, or any combination thereof.

### Predominantly Hydrophilic Topical Formulations

Also provided herein are topical formulations comprising about 0.001 to 10% by weight of detomidine or a salt thereof, and, a carrier that is suitable for topical administration to a human subject's skin, wherein the carrier comprises a hydrophobic phase member and a hydrophilic phase member in a ratio of not more than 49.9%; and, wherein the detomidine is fully dissolved and chemically stable in the hydrophilic phase member.

For example, the formulations may include about 0.005 to about 10, about 0.01 to about 8, about 0.05 to about 6, about 0.05 to about 5, about 0.05 to about 3, about 0.005 to about 2, about 0.005 to about 3, about 0.003 to about 3, about 0.008 to 3, about 0.01 to about 3, about 0.01 to about 2, about 0.01 to about 1.5, about 0.01 to about 1, about 0.01 to about 2, 0.033 to about 1, 0.033 to about 0.33, about 0.08 to about 0.5, about 0.08 to about 0.2, about 0.05 to about 3, about 0.05 to about 2, about 0.05 to about 1, about 0.1 to about 3, about 0.1 to about 2.5, about 0.1 to about 2, about 0.1 to about 1.5, about 0.1 to about 1, about 0.33 to about 1, about 0.5 to about 1, or about 0.5 to about 0.75 wt % detomidine or salt thereof, or about 0.001, about 0.002, about 0.003, about 0.005, about 0.007, about 0.008, about 0.009, about 0.01, about 0.03, about 0.05, about 0.075, about 0.08, about 0.09, about 0.1, about 0.2, about 0.3, about 0.33, about 0.5, about 0.7, about 0.75, about 0.8, about 1.0, about 1.2, about 1.25, about 1.33, about 1.4, about 1.5, about 1.6, about 1.7, about 1.8, about 1.9, about 2.0, about 2.1, about 2.2, about 2.3, about 2.33, about 2.4, about 2.5, about 2.6, about 2.7, about 2.8, about 2.9, about 3, about 3.2, about 3.4, about 3.5, about 3.7, about 4, about 4.2, about 4.5, about 4.8, about 5, about 5.2, about 5.5, about 5.8, about 6, about 6.2, about 6.5, about 6.8, about 7, about 7.2, about 7.5, about 7.8, about 8, about 8.2, about 8.5, about 8.8, about 9, about 9.2, about 9.5, about 9.7, or about 10 wt % of detomidine or salt thereof.

Other characteristics of the formulations comprising hydrophobic phase member and a hydrophilic phase member in a ratio of not more than 49.9% may be the same as those described above in connection with the topical formulations that include a hydrophilic phase member and a hydrophobic phase member in a ratio of not more than 49.9%. For example, the % w/w of the detomidine or salt thereof that is present in the hydrophilic phase member; the identity of possible detomidine salts; the respective identities of the hydrophilic and hydrophobic phase members; the optional presence of an aqueous buffer as part of the hydrophilic phase member; the optional presence of inert excipients; and the optional presence of one or more additional components in order to produce the topical form (such as thickening or gelling agents, preservatives, antioxidants, permeation enhancers, emulsifying agents, emollients, or humectants); and, other characteristics that do not cause the formulations to depart from the requirement that the hydrophobic phase member and the hydrophilic phase member in a ratio of not more than 49.9%, may all be drawn from the description provided above in connection with the other disclosed formulations (i.e., those featuring a hydrophilic phase member and a hydrophobic phase member in a ratio of not more than 49.9%). To the extent applicable, the characteristics of the topical formulations that include a hydrophilic phase member and a hydrophobic phase member in a ratio of not more than 49.9% that are disclosed above are incorporated herein with respect to the present formulations in which the hydrophobic phase member and the hydrophilic phase member in a ratio of not more than 49.9%.

In the present, second type of topical formulation containing detomidine or a salt thereof in which the hydrophobic phase member and a hydrophilic phase member in a ratio of not more than 49.9%, it is the hydrophilic phase member that predominates within the carrier. For example, the hydrophobic phase member and hydrophilic phase member are present in the carrier in a ratio of not less than about 49, 47, 45, 40, 35, 30, 25, 20, 15, 12, 10, 8, 5, 3, or 2%, or the hydrophobic phase member and hydrophilic phase member may be present in the carrier in a ratio of about 49, 47, 45, 42, 40, 37, 35, 32, 30, 27, 25, 22, 20, 18, 17, 16, 15, 12, 10, 8, 5, or 2%. In particular embodiments, the hydrophobic phase member and a hydrophobic phase member are present in the carrier in a ratio not more than about 20%, or not more than about 10%.

Some formulations in which the hydrophobic phase member and a hydrophilic phase member are present in a ratio of not more than 49.9% may comprise a cream. Respective members of the formulation and excipients may be selected in order to provide the cream form. For example, the cream may include a stiffening agent, an emulsifier, and an emollient. In such embodiments, the stiffening agent may be a fatty alcohol, an ester thereof, or both. Sample embodiments are described more fully in the examples provided infra.

The present formulations in which the hydrophobic phase member and a hydrophilic phase member are present in a ratio of not more than 49.9% may comprise an oil-in-water emulsion in which porous microparticles or nanoparticles are dispersed, at least some of the pores of the microparticles or nanoparticles being loaded with detomidine or a salt thereof, a further active pharmaceutical agent, or both. Such formulations therefore represent a dispersion of the microparticles or nanoparticles. The microparticles or nanoparticles may have pores with any appropriate diameter for accepting and releasing the detomidine and any additional active agent. For example, the microparticles or nanoparticles may have an average pore diameter of about 0.05 to 0.5 μm. The microparticles or nanoparticles may be comprise poly(lactic-co-glycolic acid), poly(lactic acid), poly(l-lactic) acid, chitosan, methyl methacrylate/ethylene glycol dimethacrylate crosspolymer, a cellulose ether polymer, or a methacrylate polymer. Exemplary cellulose ether polymers for forming micro particles and nanoparticles include ethyl cellulose, hydroxypropyl methyl cellulose phthalate, hydroxypropyl methyl cellulose acetate succinate, and cellulose acetate. Exemplary methacrylate polymers include Eudragit® polymers (e.g., Eudragit® L100, Eudragit® RL100, Eudragit® RS100, or Eudragit® E). Those skilled in the art can readily identify appropriate methods for forming micro- or nanoparticles using these or other materials.

The present formulations in which the hydrophobic phase member and a hydrophilic phase member are present in a ratio of not more than 49.9% may comprise a mixture of an emulsion and a gel, commonly known as an emulgel. Emulgels, which represent gelled emulsions, can feature properties that are beneficial for dermatological use, such as being thixotropic, greaseless, easily spreadable, easily removable, emollient, nonstaining, long shelf-life, bio-friendly, transparent, and of pleasing appearance (S.P. Stanos, Topical agents for the management of musculoskeletal pain, J. Pain Sympt. Manage. (2007)). Exemplary emulgels are described infra.

The present formulations that include a hydrophobic phase member and a hydrophilic phase member in a ratio of not more than 49.9% may comprise a microemulsion gel (also referred to as a gelled microemulsion). In such systems, microemulsions may be gelled using colloidal or mesoporous silica, magnesium stearate, sodium stearyl fumarate, or another gelling agent or emulsifier. When a silicone-based emulsifier or gelling agent, the silicone-based emulsifier may be, for example, liquid alkylmethyl silicone polyether copolymer, ethoxylated and propoxylated silicone surfactant, cyclopentasiloxane, or cyclopentasiloxane with polyethylene glycol/polypropylene glycol-19/19 dimethicone. Other gelling agents include polyamide-4, polyamide-3 (e.g., OleoCraft™ HP33), polyvinyl pyrrolidone, and polyvinyl alcohol. Nonlimiting examples of oils for use in the present microemulsion gels include oleic acid, Miglyol® 810 or 812 (triglycerides of the fractionated plant fatty acids C₈ and C₁₀), and mineral oil.

The formulations according to the present disclosure that include a hydrophobic phase member and a hydrophilic phase member in a ratio of not more than 49.9% may comprise an oil-in-water emulsion. Such embodiments may take the form of a cream, for example. The hydrophobic phase member in exemplary oil-in-water cream formulations may be hydrophobic phase member comprises PEG 2 stearyl ether, steareth-21, mineral oil, or any combination thereof.

Other embodiments of the formulations having a ratio of hydrophobic to hydrophilic phase members of not more than 49.9% can be an oil-in-water gelled emulsion.

### Microparticle Dispersions

Also provided are topical formulations comprising an oil-in-water emulsion in which porous microparticles or nanoparticles are dispersed, at least some of the pores of said microparticles or nanoparticles being loaded with detomidine or a salt thereof, with or without a further active pharmaceutical agent. In some instances, an amount of detomidine or salt thereof may be incorporated within the particle itself, i.e., in a portion of the particle other than a pore thereof. Such formulations therefore represent a dispersion of the microparticles or nanoparticles. The emulsion in which the micro- or nanoparticles are dispersed need not independently contain any active pharmaceutical agent. Indeed, in certain embodiments, the present formulations include detomidine or a salt thereof only as present within the pores of the particles. The microparticles or nanoparticles may have pores with any appropriate diameter for accepting and releasing the detomidine and any additional active agent. For example, the microparticles or nanoparticles may have an average pore diameter of about 0.05 to 0.5 μm. The microparticles or nanoparticles may be comprise poly(lactic-co-glycolic acid), poly(lactic acid), poly(l-lactic) acid, chitosan, methyl methacrylate/ethylene glycol dimethacrylate crosspolymer, a cellulose ether polymer, or a methacrylate polymer. Exemplary cellulose ether polymers for forming micro particles and nanoparticles include ethyl cellulose, hydroxypropyl methyl cellulose phthalate, hydroxypropyl methyl cellulose acetate succinate, and cellulose acetate. Exemplary methacrylate polymers include Eudragit® polymers (e.g., Eudragit® L100, Eudragit® RL100, Eudragit® RS100, or Eudragit® E). Those skilled in the art can readily identify appropriate methods for forming micro- or nanoparticles using these or other materials.

### Sprayable Compositions

The present disclosure also provides sprayable compositions comprising detomidine or a salt thereof that form a transdermal patch when sprayed onto the subject's skin. Certain embodiments of such compositions are described more fully in the examples, infra.

In some embodiments, the sprayable compositions according to the present disclosure comprise about 1% detomidine or a salt thereof, about 6% povidone, about 4% povidone vinyl acetate, about 0.5% vitamin E, about 0.05% menthol, about 3% dimethyl isosorbide, about 10% acetone, about 10% ethanol, about 25.45% tetrafluroroethane and, about 40% dichlorodifluoromethane.

In other embodiments, the sprayable composition for topical administration comprises about 0.5 to about 15% of detomidine or a salt thereof, about 6% povidone, about 4% povidone vinyl acetate, about 0.5% vitamin E, about 5% dimethyl isosorbide, about 20% ethanol and, trichloromonfluoromethane.

In further examples, the sprayable composition for topical administration comprises about 1% detomidine or a salt thereof, about 6% povidone, about 4% povidone vinyl acetate, about 1% vitamin E, about 0.05 menthol, about 5% dimethyl isosorbide, about 27.5% acetone, about 27.5% ethanol, and about 27.5% trichlomonofluoromethane.

Other embodiments of sprayable formulations may comprise (a) 0.0001 to 30 weight percent of detomidine or a salt thereof; and (b) 15 to 97 weight percent isopropyl myristate, the detomidine or salt thereof being present in an effective treatment for neuropathic pain, the pharmaceutical composition being a liquid and being in a form suitable for topical administration to a human. In certain embodiments, the composition includes at least 25 weight percent isopropyl myristate, at least 40 weight percent isopropyl myristate, or about 38-60 weight percent isopropyl myristate.

In particular embodiments, the composition includes at least 38 weight percent isopropyl myristate, and at least 40 weight percent of the composition is formed from water, ethanol, isopropyl alcohol, or any combination thereof.

The composition may alternatively comprise 38-60 weight percent isopropyl myristate, 1.5-10 weight percent water, and 35-60 weight percent ethanol.

### Detomidine Cloth or Wipe

The present disclosure also provides wipes (which may alternatively be referred to as cloths) for the treatment of pain comprising about 0.25 to about 6% w/w of detomidine or a salt thereof. The wipe may be provided within a pouch resistant to leakage. For example, the pouch may comprise an inner lining of linear low density polyethylene (LLDPE).

The present wipes may further comprise an alcohol, a buffering agent, and water. The alcohol may be, for example, ethanol. In such embodiments, the wipes may further comprise propylene glycol. Any suitable buffering agent may be used, and in certain embodiments, the buffering agent is suitable for maintaining a pH of about 3.5 to about 6. For example, the buffering agent may comprise citric acid and sodium citrate to maintain a pH of about 3.5 to about 6. In another embodiment, the buffering agent is citric acid and tromethamine in order to maintain a pH of about 3.5 to about 6. In specific embodiments, the buffering agent maintains a pH of about 4.5.

In the present wipes, the alcohol and water may be present in a weight ratio of, for example, about 40:60 to about 60:40.

When the wipes include an alcohol, a buffering agent, and water, the alcohol may be present at about 53.7 to about 57.3% w/w, the buffering agent may be included at about 0.2 to about 0.5% w/w, and the water may be added to 100% w/w.

When the wipes include an alcohol, a buffering agent, water, and propylene glycol, the alcohol may be included at about 53.7 to about 57.3 w/w, the propylene glycol may be present at about 2 to about 4% w/w, the buffering agent may be at about 0.2 to about 0.5% w/w, and the water may be added to 100% w/w.

In certain embodiments, the present wipes further comprise a polymer system comprising a hydrophobic polymer in combination with a hydrophilic polymer. For example, the polymer system may include a butyl ester of polyvinylmethylether/maleic anhydride copolymer and a polyvinyl pyrrolidone.

In wipes that comprise a polymer system, a buffering agent may be included hat is suitable for maintaining a pH of about 3.5 to about 6. For example, the buffering agent may comprise citric acid and sodium citrate to maintain a pH of about 3.5 to about 6. In another embodiment, the buffering agent is citric acid and tromethamine in order to maintain a pH of about 3.5 to about 6. In specific embodiments, the buffering agent maintains a pH of about 4.5. Such wipes may also include an alcohol, water, or both. For example, the wipes that comprise a polymer system may include ethanol and water in a weight ratio of about 50:50 to about 60:40.

In certain embodiments of the wipes that comprise a polymer system the alcohol may be present at about 53.7 to about 57.3% w/w, the buffering agent may be at about 0.2 to about 0.5% w/w, the polyvinyl pyrrolidone may be present at about 4% w/w, the butyl ester of polyvinylmethylether/maleic anhydride copolymer may be included at about 0.25% w/w, and the water may be added to 100% w/w.

### Topical Liposomal Compositions

Also disclosed herein are topical liposomal compositions for delivering a drug comprising lipid components between 5% to 30% w/w, wherein said lipid components comprise phospholipid and cholesterol; wherein said the drug is detomidine and has a particle size of about 100 nm; a solvent; and a plurality of additives selected from the group consisting of penetration enhancers, emulsifiers, antioxidants, buffering agents, pH adjusting agents, antimicrobial preservatives, one or more gelling agents, thickening agent, emollient, skin conditioner, viscosity increasing agent, film-forming agent, absorbent, water, and combinations thereof.

The amount of detomidine in the composition may range from about 0.01% to about 10% by weight, for example, about 0.1% to about 1% by weight, based on the total weight of the composition.

In certain instances, the phospholipid is selected from a group consisting of phosphatidylcholine (PC) distearoyl phosphatidylcholine (DSPC), dipalmitoyl phosphatidylcholine (DPPC), dimirystoyitoyl phosphatidylcholine (DMPC), dilauroyl phosphatidylcholine (DLPC), soya phosphatidylcholine (SPC), egg phosphatidylcholine (EPC), hydrogenated soya phosphatidylcholine (HSPC), hydrogenated egg phosphatidylcholine (HEPC); and combinations thereof.

The liposomes in the present topical liposomal compositions may have an average diameter of less than about 150 nm.

The plurality of additives may comprise an antioxidant selected from the group consisting of vitamin E, butylated hydroxyl anisole (BHT), and mixtures thereof. In certain instances, the plurality of additives may comprise a penetration enhancer selected from the group consisting of oleic acid, propylene glycol, and combinations thereof. In some embodiments, the plurality of additives may comprise an antimicrobial preservative selected from the group consisting of methyl parabene (MP), propyl parabene (PP), phenol, and combinations thereof.

The solvent may comprise, for example, at least two solvents for dissolving the lipid components, wherein the at least the two solvents are propylene glycol and glycerol in a combined amount of about 1% to about 25% by weight, based on the total weight of the composition. In other instances, the solvent comprises dimethyl sulfoxide (DMSO) and/or alcohol.

The topical liposomal composition according to the present disclosure may be formulated in a form selected from the group consisting of a cream, a serum, a lotion, a gel, and combinations thereof.

Also disclosed herein are methods for preparing a topical liposomal compositions comprising dissolving lipid components in a solvent; mixing the dissolved lipid components in said solvent with a plurality of additives selected from the group consisting of a penetration enhancer, an antimicrobial preservative, an antioxidant, and combinations thereof, forming a lipid phase thereof; melting the lipid phase, forming a uniform dissolved lipid phase; dissolving and mixing detomidine or a salt thereof into the solvent in the uniform lipid phase, forming an admixture thereof; heating the admixture; dissolving an emulsifier into a solution, preparing an aqueous phase thereof; heating the solution in the aqueous phase; and mixing and homogenizing the admixture and the solution in the aqueous phase to obtain the topical liposomal composition; and adding one or more of a gelling agent, thickening agent, emollient, skin conditioner, viscosity increasing agent, film-forming agent, or absorbent.

Pursuant to such methods, the solution may comprise, for example, triethanolamine in water or triethanolamine in a buffer solution.

For the step of heating the lipid phase and aqueous phase, the temperature may be, for example, between about 50-75° C.

The solvent may comprise at least two solvents for dissolving the lipid components. For example, the solvent may be propylene glycol and glycerol in a combined amount of about 1% to about 25% by weight based on the total weight of the composition. In other instances, the solvent comprises dimethyl sulfoxide (DMSO) or alcohol. The amount of the dimethyl sulfoxide (DMSO) or alcohol may range from about 1% to about 20% by weight of the total weight of the composition.

In the present methods, the liposomes in the topical liposomal composition may have an average diameter less than about 150 nm.

The lipid components may comprise phospholipid and cholesterol. In such instances, the phospholipid may be selected from the group consisting of phosphatidylcholine (PC), distearoyl phosphatidylcholine (DSPC), dipalmitoyl phosphatidylcholine (DPPC), dimirystoyitoyl phosphatidylcholine (DMPC), dilauroyl phosphatidylcholine (DLPC), soya phosphatidylcholine (SPC), egg phosphatidylcholine (EPC), hydrogenated soya phosphatidylcholine (HSPC), hydrogenated egg phosphatidylcholine (HEPC) and combinations thereof. The amount of the lipid component may be between about 5% to about 30% w/w.

In the present methods, the plurality of additives may comprise an antioxidant. The antioxidant may be, for example, vitamin E, butylated hydroxyl anisole (BHT), or a mixture thereof.

In certain embodiments, the plurality of additives may comprise a penetration enhancer selected from the group consisting of oleic acid, propylene glycol, and combinations thereof.

The plurality of additives may also or alternatively comprise an antimicrobial preservative selected from the group consisting of methyl paraben (MP), propyl paraben (PP), phenol, and combinations thereof.

### Opaque Emulsion Gel Formulations

Also disclosed herein are topical pharmaceutical compositions in the form of an opaque emulsion-gel comprising
(a) 1.2-4% (w/w) of detomidine monohydrate or a salt thereof,
(b) 0.5-2% (w/w) of a saturated or unsaturated C₁₀-C₁₈ fatty alcohol selected from the group consisting of stearyl alcohol, myristyl alcohol, lauryl alcohol and oleyl alcohol;
(c) at least 40% (w/w) of water,
(d) 10-30% (w/w) of at least one C₂-C₄-alkanol,
(e) 3-15% (w/w) of at least one glycol solvent selected from the group consisting of 1,2-propanediol and polyethylene glycol (200-20000),
(f) 0.5-5% (www) of at least one gelling agent selected from the group consisting of carbomers,
(g) 2-8% (w/w) of at least one liquid lipid forming the oily phase of the emulsion-gel,
(h) 1-3% (w/w) of at least one non-ionic surfactant, and
(i) a basic agent to adjust the pH of the total composition to 6-9.

In some embodiments, the saturated or unsaturated C₁₀-C₁₈ fatty alcohol of (b) is oleyl alcohol.

The present opaque emulsion-gel compositions may comprise
(a) 1.5-3.5% of detomidine monohydrate or a salt thereof,
(b) 0.5-2% oleyl alcohol,
(c) 45-75% of water,
(d) 10-30% of ethanol, isopropanol, or mixtures thereof
(e) 3-12% of 1,2-propanediol,
(f) 0.7-3% of at least one gelling agent selected from the group consisting of carbomers,
(g) 3-7% of at least one liquid lipid forming the oily phase of the emulsion-gel,
(h) 1-3% of at least one non-ionic surfactant, and
(i) 0.5-2% of diethylamine to adjust the pH of the total composition to 6.5-8.5.

In such compositions, the oleyl alcohol (b) may be present in an amount of from 0.6 up to 1.2% of the total composition.

In any of the preceding opaque emulsion-gel compositions, the component (d) may be isopropanol. In some embodiments, the liquid lipid (g) is selected from the group consisting of hydrocarbons, glycerides, esters of fatty acids, and any mixture thereof. In certain embodiments, the non-ionic surfactant (h) is selected from the group consisting of esters of fatty acids with monohydroxy or polyhydroxy compounds and poly(oxyethylated) surfactants, and any mixture thereof.

### Methods of Using the Formulations

Topical administration of the formulations once- or twice-daily to a subject for up to four days can result in a blood plasma concentration in the subject that is less than that required to achieve a systemic therapeutic effect of the detomidine or salt thereof. According to one embodiment, topical administration of the formulations once- or twice-daily to a subject for at least four days results in a blood plasma concentration in the subject that remains less than that required to achieve a systemic therapeutic effect of the detomidine or salt thereof. Preferably, the topical administration can continue for weeks, months, or longer while maintaining a sub-therapeutic systemic blood plasma concentration.

The present invention also provides methods for treating pain in a subject in need thereof comprising topically administering to the subject an effective amount of a topical formulation according to any one of the embodiments disclosed herein. The present disclosure also provides methods for providing prolonged, non-systemic treatment for pain in a subject in need thereof comprising topically administering to the subject a formulation according to any one of the embodiments disclosed above.

In certain embodiments, the pain treated according to the methods of the present invention is neuropathic pain. The pain treated according to the present methods may alternatively be erythromelalgia. In certain embodiments, the neuropathic pain is postherpetic neuralgia. In certain embodiments, the neuropathic pain is diabetic peripheral neuropathy.

The pain for which any of the above methods provide treatment can be any type of pain for which topical treatment is relevant, whether acute or chronic. For example, the pain for which treatment is provided using the present methods may be somatic (caused by the activation of pain receptors in either the body surface or musculoskeletal tissues, such as post-surgical pain), visceral (caused by damage or injury to internal body structures or organs), or neuropathic (postherceptic neuralgia and diabetic neuropathy representing examples). Exemplary types of pain that can be treated according to the present methods include carpal tunnel syndrome, abdominal pain, hip pain, knee and other joint pain, pain deriving from piriformis syndrome, back pain, neck or shoulder pain, acute or chronic muscle pain, trigeminal neuralgia, postherceptic neurolgia, sciatica pain, arachnoiditis (spinal pain), pain from complex regional pain syndrome, phantom limb pain diabetes-related nerve pain (neuropathy), pain deriving from depression or anxiety, and pain from compartment syndrome.

The present disclosure also provides methods for providing treatment for rosacea in a human subject in need thereof comprising topically administering to the human subject a topical formulation according to any one of the embodiments disclosed herein. For example, the the topical administration may be to areas of the subject's skin that include rosacea discoloration.

In accordance with the presently disclosed methods, the topical administration may be performed using conventional techniques. For example, the administration may be performed by delivering an aliquot of the formulation to a physician's or subject's hand, which is used to smear and then rub the formulation onto an area of skin on the body part for which treatment is desired. In the case of a spray patch, the formulation may be sprayed using any suitable mechanism, such as an aerosol, mister, spray bottle, or the like. The formulation may be topically administered in the chosen manner on a once-daily or twice-daily basis. When the method comprises applying the formulation on a twice-daily basis, appropriate temporal spacing between applications should be used. For example, if the subject is awake for a 16 hour period of the day, then a first application can be performed in the morning, and a second application can be performed in the evening, for example, prior to retiring to bed.

The present invention also includes modified versions of each topical formulation described herein, wherein the detomidine or a salt thereof is replaced with a different active ingredient, in the same amounts and concentrations described herein for detomidine or a salt thereof, and to methods of topically administering and treating pain described herein using such formulations. Suitable active ingredients that can be used in the compositions of the present invention instead of detomidine or a salt thereof include other α-2-adrenergic agonists. Preferred α-2-adrenergic agonists include romifidine, brimonidine, dexmedetomidine, and salts thereof. Another preferred α-2-adrenergic agonist is clonidine or a salt thereof, with clonidine hydrochloride being particularly preferred.

### EXAMPLES

The following examples are set forth so as to provide those of ordinary skill in the art with a complete disclosure and description of how the methods, compositions, and components claimed herein are made and evaluated, and are intended to be purely exemplary of the invention and are not intended to limit the scope of what the inventors regard as their invention.

### Example 1—Foam

A foam formulation that features a carrier comprising a hydrophilic phase member and a hydrophobic phase member in a ratio of not more than 49.9% is prepared from components as listed in Table 1.

TABLE 1

| Ingredient | Range (% w/w) | Function |
|---|---|---|
| Caprilic capric trigliceride | 50-70% | Emollient |
| Propylene glycol | 0-15% | Humectant |
| Hexylene glycol | 0-15% | Humectant |
| Purified water | 0-15% | |
| Lecithin | 5-15% | Surfactant |
| Glyceryl monooleate | 0-5% | Low HLB surfactant |
| PVP K-90 | 0-4% | Gelling agent |
| Preservative | 0-1.0% | |

Detomidine is dissolved in the hydrophilic phase member at up to 40% w/w, producing a final concentration of detomidine in the foam of up to 10% by weight.

A further foam formulation comprising a hydrophilic phase member and a hydrophobic phase member in a ratio of not more than 49.9% is prepared from components as listed in Table 2.

TABLE 2

| Ingredient | Range (% w/w) | Function |
|---|---|---|
| Mineral oil | 4-10% | |
| Isopropyl Myristate | 4-10% | Emollient |
| Glyceryl monooleate | 0-1% | Low HLB |
| | | surfactant |
| GlycStearyl alcohol | 0-1% | Low HLB |
| | | surfactant |
| Urea | 0-15% | |
| Xanthan gum | 0-1% | Gelling agent |
| Methocel K100M | 0-1% | |

Detomidine is dissolved in the hydrophilic phase member at up to 10% w/w, producing a final concentration of detomidine in the foam of up to 5% by weight.

### Example 2—High Oil Content Cream

A cream formulation that features a carrier comprising a hydrophilic phase member and a hydrophobic phase member in a ratio of not more than 49.9% is prepared from components as listed in Table 3 (hydrophobic phase A and hydrophilic phase B are listed separately).

TABLE 3

| Ingredient | Range (% w/w) | Function |
|---|---|---|
| Phase A | | |
| Mineral Oil | 60-80% | Emollient |
| Isopropyl Isostearate (Crodamol IPIS) | 30-40% | Emollient |
| Isostearyl Isostearate (Crodamol ISIS) | 30-40% | Emollient |
| Brij S2 | 0-5% | Surfactant |
| Brij S721 Crodex M | 0-5% | Surfactant |
| Crodex M | 0-15% | Surfactant |
| Crodafos CES | 0-15% | Surfactant |
| Phase B | | |
| Glycerine | 0-5% | Humectant |
| Propylene Glycol | 0-5% | Humectant |
| Purified water | Ad. 100% | |
| API | 0-1% | API |

Detomidine is dissolved in phase B at up to 20% w/w, ultimately producing a final concentration of detomidine in the cream of up to 6% by weight. Phases A and B are separately heated to 70-75° C. Phase A is added to phase B with paddle stirring of about 500 RPM. The two phases are then homogenized with Ultra Turax 9000-10000 RPM, 1 min/100 g, and the resulting material is stirred with paddle at about 200 RPM until cooled to room temperature.

### Example 3—High Water Content Cream

A cream formulation that features a carrier comprising a hydrophilic phase member and a hydrophobic phase member in a ratio of not more than 49.9% is prepared from components as listed in Table 4.

TABLE 4

| Agent | % w/w |
|---|---|
| Detomidine | 0.001-10 |
| Stiffening agent | 5-10% |
| Emulsifier | 4-10%. |
| Emollient | 1-10% |
| Viscosity modifier | 1-3% |
| Preservative | 0.1-3% |
| Vehicle/Water/Buffer | Add to 100% |

In a particular embodiment of the preceding cream formulation, the detomidine is present in the cream in a final concentration of 0.1% by weight.

### Example 4—Oil-in-Water Emulsion with Dispersed Microparticles

Microparticle preparation. Detomidine-loaded microparticles are prepared by combining 15% w/w detomidine with 40% w/w dimethicone 200, 350 cst, and 45% w/w microparticles. The microparticles are Microsponge® porous methyl methacrylate/ethylene glycol dimethacrylate crosspolymer microparticles (Advanced Polymer Systems, Inc., Redwood City, Calif.), having a weight average particle diameter of 20 a surface area of 225 m²/g, and a pore volume of 1 cm³/g.

Combination with emulsion. The detomidine-loaded microparticles are combined with an oil-in-water emulsion as follows using Parts I-VII (Part V representing the microparticles):

| Part I | |
|---|---|
| Purified water, USP | 27.67 |
| Carbomer 940, NF (Carbopol 940) | 0.40 |
| Part II | |
| Glycerin, USP (Emery 912) | 7.00 |
| PEG-8 (Carbowax-400) | 10.00 |
| Part III | |
| Detomidine | 1.50 |
| Part IV | |
| Polysorbate 80 (Tween 80) | 4.50 |
| Sorbitan oleate (Span 80) | 3.50 |
| Oetyl hydroxy stearate (Wickenol-171) | 12.00 |
| Methyl gluceth-20 (Glucam E-20) | 5.00 |
| Stearic acid, NF (Emersol 132 Triple press) | 1.50 |
| Part V | |
| Microparticles | 23.33 |
| Part VI | |
| Triethanolamine, 99% | 0.40 |
| Purfied water, USP | 1.00 |
| Part VIII | |
| Propylene glycol, diazolidinyl urea, methylparaben, | 1.20 |
| and | |
| propylparaben blend (Germaben II) | |
| Purified water, USP | 1.00 |

The water of Part I is weighed in a suitable container and the carbomer 940 added and mixed for about 30 minutes, then transferred to a mixing vessel. The ingredients of Part II are added to the mixture, and the mixture heated to 75-80° C. The free fluorouracil of Part III is then added to the mixture with good mixing for 5-10 minutes, forming the aqueous phase. The ingredients of Part IV are mixed and heated to 75-80° C., forming the oil phase. The oil phase is slowly added to the aqueous phase under increased mixing, forming an oil-in-water emulsion, and the heating terminated. Part V i added very carefully to the mixture of Parts I through IV under mixing, until complete dispersion is achieved. The ingredients of Part VI is pre-mixed and added with mixing, and the temperature lowered. At 40° C., the ingredients of Part VII are pre-mixed and added to the mixture, and the temperature is lowered further to 30° C. or below.

In another embodiment, the oil-in-water emulsion that is combined with the microparticles does not contain any active pharmaceutical ingredient.

### Example 5—Spray Patch

A formulation that can be sprayed onto a subject's skin in order to form a transdermal patch is prepared using the materials shown in Table 5.

TABLE 5

| | Range | |
|---|---|---|
| Ingredient | (% w/w) | Function |
| Detomidine | 0-15% | API |
| API 2 (optional) | 0-15% | API |
| Dicalcium Phosphate | 15-40% | Filler |
| Polyvinyl Alcohol | 10-25% | Gelling agent |
| White Petrolatum | 0-10% | Oil base |
| Span 40 | 0-5% | Surfactant |
| Span 60 | 0-5% | Surfactant |
| Preservative | 0-1% | Preservative |

In a particular embodiment, the detomidine is included in the formulation in an amount of 0.1% w/w.

A further embodiment of a spray patch formulation is prepared using the materials shown in Table 6.

TABLE 6

| Ingredient | Range (% w/w) | Function |
|---|---|---|
| Eudragit RL PO | 0-20% | Gelling agent |
| DynamX | 0-20% | Gelling agent |
| Klucel LF | 0-20% | Gelling agent |
| Triethyl Citrate | 0-7% | Plastisizer |
| Ethanol | 60-90% | Solvent |
| Detomidine | 0-5% | API |

### Example 6—Micro-Emulsion Gels with Silicone-Based Gelling Agents

Micro-emulsion gels (versions a-i) using silicone-based gelling agents are provided in Table 7.

TABLE 7

| | Example | | | | | | | | |
|---|---|---|---|---|---|---|---|---|---|
| | a | b | c | d | e | f | g | h | i |
| Xiameter ® PMX-245 | 78.0 | 14.3 | 15.5 | 4.5 | — | 7.4 | 27.5 | — | — |
| (cyclopentasiloxane) | | | | | | | | | |
| Dowsil ™ BY 11-030 | 20.1 | — | — | — | — | — | — | — | — |
| (50% dispersion of a high | | | | | | | | | |
| molecular weight silicone | | | | | | | | | |
| surfactant polyoxyethylene/ | | | | | | | | | |
| polyoxypropylene | | | | | | | | | |
| copolymer) in | | | | | | | | | |
| XIAMETER ® PMX-0245 | | | | | | | | | |
| cyclopentasiloxane) | | | | | | | | | |
| Dowsil ™ ES-5226 | — | — | — | 20.0 | 15.9 | 13.3 | 28.0 | 18.9 | 43.4 |
| (ethoxylated and | | | | | | | | | |
| propoxylated silicone | | | | | | | | | |
| surfactant) | | | | | | | | | |
| Dow Corning ® 5200 | — | 14.4 | 15.5 | — | — | — | — | — | — |
| (Lauryl PEG/PPG-18/18 | | | | | | | | | |
| Methicone - liquid alkylmethyl | | | | | | | | | |
| silicone polyether copolymer) | | | | | | | | | |
| Transcutol ® | — | 43.3 | 38.1 | 18.0 | — | — | 42.1 | — | — |
| (2-(2-Ethoxyethoxy)ethanol) | | | | | | | | | |
| Propylene Glycol | — | 27.9 | 31.0 | 14.6 | — | — | 2.4 | — | — |
| Diisopropyl Adipate | — | — | — | — | 13.3 | 14.8 | — | 11.1 | 16.6 |
| Miglyol ® 810 | — | — | — | 31.7 | 13.3 | — | — | 7.8 | 8.3 |
| (triglycerides of the fractionated | | | | | | | | | |
| plant fatty acids C₈ and C₁₀) | | | | | | | | | |
| Water | 2.0 | — | — | 11.2 | 57.6 | 64.4 | — | 62.3 | 31.7 |

Detomidine is dissolved in the hydrophilic phase member (including propylene glycol, water, or both) at up to 40% w/w, producing a final concentration of detomidine in the micro-emulsion gels of up to 10% by weight.

### Example 7—Emulgel

An emulgel comprising a hydrophobic carrier member and a hydrophilic carrier member in a ratio of not more than 49.9% is prepared using the materials listed in Table 8.

TABLE 8

| | Range | |
|---|---|---|
| Ingredient | (% w/w) | Function |
| Carbomer 974 | 0-2% | Gelling agent |
| Macrogol cetostearyl ether | 0-10% | surfactant |
| (Cetomacrogol ™ 1000) | | |
| Oleyl alcohol | 0-10% | Pentration enhancer |
| Liquid paraffin | 0-20% | emollient |
| Isopropyl alcohol | 1-10% | Co-Solvent/penetration |
| | | enhancer |
| Cocoyl caprylocaprate | 0-20% | Emollient |
| (Cetiol ® LC) | | |
| Diethylamine | 0-1% | Gel—Alkalizing agent |
| Purified Water | Ad. 100% | |

Detomidine is dissolved in the hydrophilic phase member at up to 8% w/w, producing a final concentration of detomidine in the emulgels of up to 6.5% by weight.

### Example 8—Oil-Based Micro-Emulsion Gels

Micro-emulsion gels comprising a hydrophilic carrier member and a hydrophobic carrier member in a ratio of not more than 49.9% are prepared using the materials listed in Table 9.

TABLE 9

| | Amount | |
|---|---|---|
| Component | (wt %) | Component Chosen From |
| Water | 0-10% | |
| Solvent/co- | 0-20% | Cremophor 35EL, 50 polyoxyl |
| solvent | | stearate, hydrogenated castor oil, |
| | | Solutol 15HS (Macrogol (15)- |
| | | hydroxystearate), Co-S = Ethanol |
| Oil | 80-100% | medium-chain triglycerides (MCT), |
| | | e.g., Miglyol ® 810/812, with Benzyl |
| | | Alcohol (BA) content as low as |
| | | possible |

Specific embodiments making use of the preceding components are listed below in Table 10.

TABLE 10

| Oil (BA/ | Solvent/ | | |
|---|---|---|---|
| MCT | Co-Solvent | | |
| Ratio) | Ratio | Surfactant | Cosurfactant |
| 1/0 | 1/1 | Cremophor EL | EtOH |
| 6/1 | 1/1 | Cremophor EL | EtOH |
| 3/1 | 1/1 | Cremophor EL | EtOH |
| 1/1 | 1/1 | Cremophor EL | EtOH |
| 3/1 | 1/1 | Solutol HS15 | EtOH |
| 3/1 | 1/1 | Hydrogenated | EtOH |
| | | castor oil 15 | |
| 3/13 | 1/0 | Hydrogenated | — |
| | | castor oil 15 | |
| 3/1 | 3/1 | Hydrogenated | EtOH |
| | | castor oil 15 | |
| 3/1 | 1/3 | Hydrogenated | EtOH |
| | | castor oil 15 | |

Detomidine is dissolved in the hydrophilic phase member at up to 8% w/w, producing a final concentration of detomidine in the micro-emulsion gels of up to 2.5% by weight.

In further embodiments, the materials listed in Table 11 are used to form micro-emulsion gels comprising a hydrophilic carrier member and a hydrophobic carrier in a ratio of not more than 49.9%.

TABLE 11

| | Amount | |
|---|---|---|
| Component | (wt %) | |
| Water | 0-10% | |
| Solvent/ | 30-40% | span + lutro |
| co-solvent | | 1127/IPA |
| Oil | 60-70% | oleic acid |

Detomidine is dissolved in the hydrophilic phase member at up to 8% w/w, producing a final concentration of detomidine in the micro-emulsion gels of up to 0.8% by weight.

In further embodiments, the materials listed in Table 12 are used to form micro-emulsion gels comprising a hydrophilic carrier member and a hydrophobic carrier in a ratio of not more than 49.9%.

TABLE 12

| | Amount | Component |
|---|---|---|
| Component | (wt %) | Chosen from |
| water | 0-10% | |
| Solvent/ | 30-70% | Lecithin/ |
| co-solvent | | Absolute alcohol, |
| | | propylene glycol |
| Oil | 20-70% | Captex ®-200, |
| | | Captex ®-355 |

Detomidine is dissolved in the hydrophilic phase member at up to 8% w/w, producing a final concentration of detomidine in the micro-emulsion gels of up to 0.8% by weight.

### Example 9—Water-Based Micro-Emulsion Gels

A micro-emulsion gel comprising a hydrophobic carrier member and a hydrophilic carrier member in a ratio of not more than 49.9% is formed using the materials listed in Table 13.

TABLE 13

| | Amount |
|---|---|
| Component | (wt %) |
| Water | to 100 |
| Polychol ™ 15 | 30 |
| (PEG-15 lanolin alcohol) | |
| Mineral oil (25 cS at 25°) | 14 |
| Crodamol ™ IPM | 7 |
| (Isopropyl myristate) | |
| Super Hartolan ™ | 5 |
| (Lanolin Alcohol) | |
| PEG-200 | 5 |
| Perfume, Preservative | qs |

Detomidine is dissolved in the water phase member at up to 8% w/w, producing a final concentration of detomidine in the micro-emulsion gel of up to 3% by weight.

The oil and water phases are heated separately to 80-85° C. Water phase is added to the oil phase with careful stirring to avoid aeration. Stirring is continued as combination cools to 65-70° C.

### Example 10—Detomidine-Loaded Microparticles or Nanoparticles

Chitosan Microparticles. Detomidine in the form of solid particles (2 wt %) is incorporated into high viscosity chitosan solutions [3 wt % solutions of Protasan UP B 80/500 (FMC Biopolymers Inc.; 755 cps apparent viscosity) in 2.1 wt % glycolic acid and 0.03 wt % sodium hydroxide] in the presence of soybean oil (17 wt %) by vigorous mixing to form a matrix. The viscosity of the matrix is initially 215,000 cps as measured on a Brookfield LVT viscometer at 25° C. with appropriate spindle at 1.5 rpm. The emulsion is then mixed with a poly(acrylic acid) solution (0.5 wt %) at pH 6.3 and homogenized to make a gel containing detomidine microparticles of size below 10 microns.

PLGA Microparticles. In a separate embodiment, PLGA microparticles containing detomidine are prepared by a oil/water solvent evaporation technique as previously described (Haddadi et al. 2006). Detomidine is dissolved in ethanol and added into a 16% polymer solution in dichloromethane. The drug-polymer mixture is mixed thoroughly and emulsified into 5 ml of 0.5% w/v PVA in phosphate buffer solution (PBS) using a microtip sonicator (Heat Systems Inc., USA). This emulsion is slowly transferred into 30 ml of 0.5% w/v of PVA solution and stirred for 3 h in order to evaporate the solvent. The microparticles are then collected by ultracentrifugation (10 000 rpm for 10 min at 4 C) (IEC, B-20A ultracentrifuge.

PLGA Nanoparticles. Detomidine-loaded nanoparticles are prepared by emulsion-diffusion-evaporation method with slight modification [18, 19]. Briefly, 50 mg of PLGA and detomidine (10% w/w of polymer) are dissolved in 2.5 ml of ethyl acetate at room temperature. The organic phase is then added to 5 ml of an aqueous phase containing the 2% w/v PVA as a stabilizer to form stable emulsion. The resulting o/w emulsion is stirred at 1000 rpm for 20 min before sonication for 60 sec at 1.0 cycle at 60% of amplitude. The resulting emulsion is poured into 25 ml of water with constant stirring to diffuse and evaporate the organic solvent completely. This results in nanoprecipitation and formation of nanoparticles.

### Example 11—Emulsions With Detomidine HCl

Formulation trials were conducted in order to develop topical formulation options with detomidine HCl at 1% inclusion for analgesic application. The primary aim of the formulations generated was to create a high thermodynamic activity for the API reducing its solubility in the total formulation, or in the portion of formulation in which it resides.

Experimental. Several formulations were trialed with the formulations based on the following themes, all trying to increase the thermodynamic activity of the detomidine HCl by reducing the water phase % or oiling out the active:
W/O microemulsions based on Cithrol 10GTIS, including gelled versions
High internal phase O/W emulsions based on liquid crystal emulsifiers
W/O emulsions containing API in one portion of the emulsion diluted by a further placebo portion of the same formulation
Ringing gel microemulsions using Brij 0 types or Croduret 40
O/W Microemulsions, oiled out API and gelled with OleoCraft HP33

Detomidine HCl was supplied in neat powder form and as 20% solutions in glycerine and propylene glycol (Teva Pharmaceuticals, Israel). Rheology testing used the following method: stress controlled rheometer, Discovery HR-3, TA Instruments. Amplitude sweep at a frequency 1.0 Hz, Oscillation torque 0.1-100,000 μNm. Temperature 20.0° C. Geometry, 40 mm parallel cross hatched plate

Microscopy was carried out on an Olympus BX-51 optical microscope with polarizers.

Certain excipients used were supplied by Croda, UK, chemical names listed in Table 14.

TABLE 14

| | | |
|---|---|---|
| Arlamol PS11E | PPG-11 Stearyl Ether | PhEur |
| Brij L9 | Macrogol Lauryl | PhEur/ NF |
| | Ether PhEur | |
| Brij O20 | Macrogol Oleyl | PhEur |
| | Ether PhEur | |
| | (PEG 20 Oleyl Ether) | |
| Brij O5 | Macrogol Oleyl | PhEur |
| | Ether PhEur | |
| | (PEG 5 Oleyl Ether) | |
| Brij S2 | Macrogol Stearyl | PhEur/ NF |
| | Ether PhEur | |
| | (PEG 2 Stearyl Ether) | |
| Brij S721 | Steareth-21 | FDA IIG |
| Cithrol DPHS | PEG-30 | PhEur |
| | Dipolyhydroxystearate | |
| Crodafos CES | Cetearyl alcohol (and) | None |
| | cetheth-10 phosphate | |
| | (and) dicetylphosphate | |
| Crodamol IPIS | Isopropyl Isostearate | FDA IIG |
| Crodamol ISIS | Isostearyl Isostearate | NF in progress |
| Crodex M | Cetearyl alcohol (and) | PhEur pending |
| | potassium cetyl phosphate | |
| Croduret 40 | Macrogol Hydroxystearate | PhEur/ NF |
| | PhEur (Polyoxyl 40 | |
| | Hydrogenated Castor | |
| | Oil NF) | |
| Glycerox 767HC | Macrogol 6 Glycerol | PhEur/ NF |
| | Caprylocaprate PhEur | |
| | (Caprylocaprroyl | |
| | Polyoxyglycerides NF) | |
| NatraSense AG810 | Lauryl Alkyl Polyglucoside | None |
| | (70%) and Water (30%) | |
| OleoCraft HP33 | Polyamide-3 | None |
| Super Refined | Dimethyl Isosorbide | FDA IIG |
| Arlasolve DMI | | |
| Super Refined | Isopropyl Myristate | PhEur/ NF |
| Crodamol IPM | | |
| Synperonic PE/ L44 | Poloxamer 124 | FDA IIG |
| Xiameter PMX 245 | Cyclopentasiloxane | PhEur/ NF |
| | (ex Dow Corning) | available |

W/O Microemulsions. Water in oil microemulsions were trialled as placebos, these formulations had low water content, allowing for greater saturation of the detomidine HCl in the solubilizing aqueous/propylene glycol phase.

Formulation 3.1.1

Phase A

Cithrol 10GTIS 30.0%

Arlamol HD 20.0%

Xiameter PMX 245 25.0%

Crodamol IPIS 5.0%

Phase B

Propylene Glycol 10.0%

Deionised Water 10.0%

Method. Blend phase A with paddle stirring, once clear add phase B with paddle stirring. Formulation 3.1.1 was not optically transparent so a slight variant was manufactured, formulation 3.1.2.

Formulation 3.1.2

Phase A

Cithrol 10GTIS 30.0%

Arlamol HD 25.0%

Xiameter PMX 245 20.0%

Crodamol IPIS 5.0%

Phase B

Propylene Glycol 10.0%

Deionised Water 10.0%

Method. Blend phase A with paddle stirring, once clear add phase B with paddle stirring.

Formulation 3.1.2 was optically transparent, however, the aesthetics of the formulation were deemed to be too sticky/tacky for this application so no further work was carried out in this area.

High Internal Phase O/W Emulsions. Oil in water formulations (3.2.1-3.2.8, shown below in Table 15) with high internal oil phase (˜70%) were formulated with liquid crystal forming emulsifiers. As with formulations from 3.1 the overall aim of the formulation was to reduce the quantity of the solubilizing water phase.

TABLE 15

| Formulation | 3.2.1 | 3.2.2 | 3.2.3 | 3.2.4 | 3.2.5 | 3.2.6 | 3.2.7 | 3.2.8 |
|---|---|---|---|---|---|---|---|---|
| Phase A | | | | | | | | |
| Mineral | 70.0% | 70.0% | 70.0% | | | 35.0% | | |
| Oil | | | | | | | | |
| Crodamol | | | | | 35.0% | 35.0% | | 35.0% |
| IPIS | | | | | | | | |
| Crodamol | | | | 70.0% | 35.0% | | | 35.0% |
| ISIS | | | | | | | | |
| Brij S2 | 7.0% | | | 7.0% | 7.0% | 7.0% | 7.0% | 7.0% |
| Brij S721 | 3.7% | | | 3.7% | 3.7% | 3.7% | 3.7% | 3.7% |
| Crodex M | | 12.0% | | | | | | |
| Crodafos | | | 12.0% | | | | | |
| CES | | | | | | | | |
| Phase B | | | | | | | | |
| Glycerine | 5.0% | 5.0% | 5.0% | 5.0% | 5.0% | 5.0% | | |
| Deionized | 14.3% | 13.0% | 13.0% | 14.3% | 14.3% | 14.3% | 14.3% | 14.3% |
| Water | | | | | | | | |
| Detomidine | | | | | | | 5.0% | 5.0% |
| HCl (20% | | | | | | | | |
| solution | | | | | | | | |
| in the | | | | | | | | |
| glycerine) | | | | | | | | |

Method. Heat phases A & B to 70-75° C., add A to B with paddle stirring (500 rpm), then homogenise with an Ultra Turrax (9,500 rpm 1 min, 100 g scale). Stir to cool with paddle stirring 200 rpm.

Formulations 3.2.1 was generated successfully producing a viscous white emulsion, formulations 3.2.2 and 3.2.3 produced emulsions that split on the stirrer and showed signs of syneresis within hours of manufacture respectively. Therefore, the formulation with the Brij S2 & S721 was taken through for further work.

Variants of 3.2.1 were made altering the oil phase from mineral oil to combinations using Crodamol ISIS and IPIS to improve the sensorial aspect of the formulations, giving a less oily feel. It was deemed that the optimum sensory was delivered by a 1:1 combination of ISIS and IPIS, therefore, 3.2.1 and 3.2.5 were tested with the API, 3.2.7 and 3.2.8 respectively.

Rheological comparisons were made for the placebo and API containing formulations, one day after manufacture and after 6 weeks storage at room temperature. The rheological plots (FIGS. 1 and 2), show the placebo and API containing comparative versions, 3.2.1/3.2.7 and 3.2.5/3.2.8, respectively.

For the mineral oil based formulation 3.2.1/3.2.7 the rheology for placebo and the API material is essentially identical, suggesting that the sensorial properties would be equivalent. There was no change after 6 weeks storage suggesting the formulation is stable at least at room temperature.

For the Crodamol IPIS/ISIS formulations 3.2.5/3.2.8 it suggests that there is a subtle difference in the sensory properties between the placebo and API variant. The stability looks acceptable as there has been no change in the rheology profile over 6 weeks.

Blended Placebo and API W/O Emulsions. A water in oil formulation was generated, the proposal for this formulation was to generate an API containing portion and placebo portion which could be blended. The purpose of the blend was to increase the activity of the API in the water phase in which it was contained.

This would have been difficult to study with actual API, so for initial investigation two inorganic white/yellow water soluble species that become coloured blue on complexing with one another were used, namely iron sulphate and potassium iron cyanate

FeSO₄+K₃Fe(CN)₆→KFeFe(CN)₆

Each species was added to the water phase of the emulsion at 0.05M.

Formulation 3.3.1

Phase A

Arlamol PS11E 1.25%

Super Refined Crodamol IPM 1.25%

Mineral oil USP 7.50%

Cithrol DPHS 1.50%

Xiameter PMX 245 3.00%

Phase B

0.05M FeSO4 85.50%

Method. Heat phase A and B to 50-60° C. Add phase B to A slowly with paddle stirring (500 rpm). Homogenise at 9,500 rpm for 1 min (100 g scale).

Formulation 3.3.2 was generated with the same recipe with the exception of using 0.05M KFeFe(CN)₆ as the Phase B.

Formulations 3.3.1 and 3.3.2 were blended at 3:1, 1:1 and 1:3 ratios and samples stored overnight at room temperature and 40° C. Initially the formulations showed no blue colouration, however, after storage overnight a blue/green colour was present. This showed that the separate water phases were blending with one another, therefore, no further work was carried out on this chassis.

Ringing Gel Microemulsion Gels. In further formulations, surfactant-based ringing gels were used as the rheology modification for gels with the detomidine HCl.

Formulation 3.4.1

Phase A

Mineral oil USP 6.00%

Brij 020 15.00%

Brij 05 7.50%

Propylene Glycol 5.00%

Deionised Water 66.50%

Method. All the ingredients were blended together and heated to 70-80° C. with paddle stirring/magnetic stirrer (100 g scale). Stirring was suspended at 45-50° C. to prevent excessive air incorporation into the gel.

Formulation 3.4.2

Phase A

Croduret 40 35.00%

Arlamol PS11E 15.00%

Deionised Water 50.00%

Method. All the ingredients were blended together and heated to 70-80° C. with paddle stirring/magnetic stirrer (100 g scale). Stirring was suspended at 45-50° C. to prevent excessive air incorporation into the gel.

Both of these formulations (3.4.1 & 3.4.2) successfully generated a gelled formulation. The sensorial properties of 3.4.1 was much tackier/stickier than the Croduret 40/Arlamol PS11E based gel.

O/W Microemulsions Gelled With OleoCraft HP-33. O/W microemulsions were generated using surfactant pairs to microemulsify the detomidine HCl when it has been oiled out of aqueous solution in its basic form after the addition of NaOH. OleoCraft HP-33 was trialled as the gelling agent, as this is generally unaffected by salt or pH conditions.

Formulation 3.5.1

Phase A

Glycerox 767HC 4.00%

Synperonic PE/L44 4.00%

Glycerine 20.00%

OleoCraft HP33 3.00%

Deionised Water 69.00%

Method. All ingredients were blended together and heated to 80-90° C. with paddle stirring/magnetic stirrer (100 g scale). Stirring was suspended at 45-50° C. to prevent excessive air incorporation into the gel.

Formulation 3.5.2 consisted of a portion of 3.5.1, with 4.00% Super Refined Arlasolve DMI to improve clarity. This slightly improved the clarity of the formulation so this formulation was carried forward for API work.

Table 16 describes Formulations 3.5.3-3.5.10

TABLE 16

| Formulation | 3.5.3 | 3.5.4 | 3.5.5 | 3.5.6 | 3.5.7 | 3.5.8 | 3.5.9 | 3.5.10 |
|---|---|---|---|---|---|---|---|---|
| Glycerox | 4.0% | 4.0% | | 4.0% | 4.0% | 8.0% | 4.0% | 4.0% |
| 767HC | | | | | | | | |
| Synperonic | 4.0% | 4.0% | 4.0% | | | | | |
| PE/L44 | | | | | | | | |
| NatraSense | | | 4.0% | 4.0% | | | 4.0% | |
| AG810 | | | | | | | | |
| Brij O5 | | | | | 4.0% | | | |
| Brij L9 | | | | | | | | 4.0% |
| Glycerine | 15.00% | 15.00% | 15.00% | 15.00% | 15.00% | 15.00% | 15.00% | 15.00% |
| OleoCraft | 3.00% | 3.00% | 3.00% | 3.00% | 3.00% | 3.00% | 2.00% | 3.00% |
| HP33 | | | | | | | | |
| 0.1M NaOH | 65.00% | 65.00% | 65.00% | 65.00% | 65.00% | 65.00% | 66.00% | 65.00% |
| Detomidine | 5.00% | 5.00% | 5.00% | 5.00% | 5.00% | 5.00% | 5.00% | 5.00% |
| HCl (20% | | | | | | | | |
| solution in | | | | | | | | |
| glycerine) | | | | | | | | |
| SR | 4.00% | 4.00% | 4.00% | 4.00% | 4.00% | 4.00% | 4.00% | 4.00% |
| Arlasolve | | | | | | | | |
| DMI | | | | | | | | |

Method. All ingredients were blended together and heated to 80-90° C. with paddle stirring/magnetic stirrer (25-50 g scale). Stirring was suspended at 45-50° C. to prevent excessive air incorporation into the gel.

In all of the formulations 3.5.3-3.5.10 (except 3.5.8), it appeared that the detomidine HCl has been successfully micro-emulsified into the formulation in its basic form. A visual inspection of the placebo verses API formulations did not show any visual differences on API addition. All the formulations formed gels, and formulation 3.5.3 was stored for 6 weeks at ambient temperature, and no loss in gel structure or API crystallization was observed. The other API containing formulations 3.5.4-3.5.10 displayed no destabilization for 1 week after manufacture.

Formulations 3.5.5 and 3.5.9 both possessed significantly greater optical transparency than the original trial, 3.5.3. Formulation 3.5.9 had less OleoCraft HP33, giving a softer gel, which could represent another aesthetic option. The NatraSense AG810 used in this product does have a yellow/orange color that could be seen in the gels. In addition, NatraSense AG810 is not a pharmaceutical excipient, so alternatives were investigated.

The formulation made in this formulation category was 3.5.10, which was optically transparent and colourless. In addition, this formulation included monographed/FDA IIG excipients, with the exception of the OleoCraft HP33. The gel was stable under storage conditions.

### Example 12—Gel Cream Formulations

Numerous gel cream formulations are prepared by selecting among components as listed below in Table 17.

TABLE 17

| Component | | |
|---|---|---|
| (INCI Name) | Function | Weight % |
| phenoxyethanol | preservative | 0.7 |
| Benzalkonium | preservative | 0.05 |
| Chloride | | |
| benzyl alcohol | preservative | 0.6 / 0.4 |
| chlorphenesin | Preservative | 0.2 / 0.27 |
| disodium edta | chelating agent | 0.1 |
| glycerin | humectant | 2 / 5 |
| tocopheryl acetate | skin | 0.2 |
| | conditioner | |
| Octyldodecyl | skin | 5.5 |
| Neopentanoate | conditioner | |
| sodium ascorbyl | skin | 0.2 |
| phosphate | conditioner | |
| sodium | skin | 0.01 / 0.05 / 0.1 |
| hyaluronate | conditioner | |
| | /Emollient | |
| Hydrolyzed | skin | 0.01 / 0.05 |
| hyaluronic acid | conditioner | |
| Pentylene Glycol | skin | 1 / 0.5 |
| | conditioner | |
| Copper Gluconate; | skin | 0.1 |
| Magnesium | conditioner | |
| Aspartate; Zinc | | |
| Gluconate | | |
| sodium hydroxide | ph adjuster | 0.7 / 0.5 / 0.38 |
| caprylyl methicone | emollient | 4 / 5.5 |
| dicaprylyl carbonate | Emollient | 0.5 |
| sodium | thickener | 2/ 1.2/1/0.7 |
| acryloyldi- | agent | |
| methyltaurate/vp | | |
| copolymer | | |
| Ammonium | thickener | 1.8 |
| acryloyldi- | agent | |
| methvitaurate/ | | |
| vp copolymer | | |
| sodium polyacrylate | Thickener | 0.3 0.4 |
| | agent | |
| Polyacrylate-13; | Viscosity | 0.5 |
| Polyisobutene; | increasing | |
| Polysorbate 20 | | |
| Methyl methacrylate | Film-forming | 2 |
| Crosspolymer | agent | |
| steareth-21 | emulsifier | 0.15 / 0.3 / 0.5 |
| glyceryl stearate | emulsifier | 0.3 /0.4 / 0.8 |
| silica | absorbent | 1 |
| aqua | vehicle | Add to 100% |

A specific embodiment representing a selection from Table 17 is provided below in Table 18.

TABLE 18

| Component | | Weight |
|---|---|---|
| (INCI Name) | Function | % |
| phenoxyethanol | preservative | 0.7 |
| Benzalkonium | preservative | 0.05 |
| Chloride | | |
| benzyl alcohol | preservative | 0.6 |
| disodium edta | chelating agent | 0.1 |
| glycerin | humectant | 5 |
| tocopheryl acetate | skin | 0.2 |
| | conditioner | |
| Octyldodecyl | skin | 5.5 |
| Neopentanoate | conditioner | |
| sodium hydroxide | ph adjuster | 0.7 |
| aqua | vehicle | Add to 100% |
| caprylyl methicone | emollient | 4 |
| sodium ascorbyl | skin | 0.2 |
| phosphate | conditioner | |
| dicaprylyl | Emollient | 0.5 |
| carbonate | | |
| sodium | Thickener | 0.3 |
| polyacrylate | agent | |
| Polyacrylate-13; | Viscosity | 0.5 |
| Polyisobutene; | increasing | |
| Polysorbate 20 | | |
| chlorphenesin | Preservative | 0.2 |
| Methyl | Film-forming | 2 |
| methacrylate | agent | |
| Crosspolymer | | |
| sodium acryloyldi- | thickener | 2 |
| methyltaurate/vp | agent | |
| copolymer | | |
| Ammonium | thickener | 1.8 |
| acryloyldi- | agent | |
| methyltaurate/vp | | |
| copolymer | | |
| steareth-21 | emulsifier | 0.15 |
| silica | absorbent | 1 |
| glyceryl stearate | emulsifier | 0.3 |
| sodium hyaluronate | skin | 0.01 |
| | conditioner | |
| | /Emollient | |
| Hydrolyzed | skin | 0.01 |
| hyaluronic | conditioner | |
| acid | | |
| Pentylene Glycol | skin | 1 |
| | conditioner | |
| Copper Gluconate; | skin | 0.1 |
| Magnesium | conditioner | |
| Aspartate; Zinc | | |
| Gluconate | | |

### Example 13—Additional Foam Formulations

Foam formulations containing detomidine HCl are prepared using components and proportions as described in Table 19.

TABLE 19

| Ingredient | Synonym | Function | % | % | % | % | % | % |
|---|---|---|---|---|---|---|---|---|
| Caprylic/Capric | MCT oil | hydrophobic | 60.9 | 60.0 | 59.0 | 60.0 | 60.0 | 56.0 |
| Triglycerides | | solvent/potent | | | | | | |
| | | solvent | | | | | | |
| Propylene glycol | | Co-solvent/poten | 10.0 | 10.0 | | | 5.0 | 5.0 |
| | | solvent | | | | | | |
| Hexylene glycol | | Co-solvent/poten | | | 10.0 | 5.0 | | |
| | | solvent | | | | | | |
| Purified water | De-ionized | Solvent | 10.0 | 10.0 | 10.0 | 10.0 | 10.0 | 10.0 |
| | Distilled water | | | | | | | |
| Potent solvent | | | — | — | — | 5.0 | 5.0 | 5.0 |
| Lecithin | Phospholipids | Surfactant | 10.0 | 10.0 | 10.0 | 10.0 | 10.0 | 10.0 |
| Stearyl alcohol | Stearyl alcohol | Stabilizer | 5.0 | 5.0 | 5.0 | 5.0 | 5.0 | 5.0 |
| Glyceryl | Glyceryl | Stabilizer | 2.0 | 2.0 | 2.0 | 2.0 | 2.0 | 2.0 |
| monostearate | monostearate | | | | | | | |
| PVP K90 | Polyvinyl | Gelling agent | 2.0 | 2.0 | 2.0 | 2.0 | 2.0 | 2.0 |
| | pyrrolidone | | | | | | | |
| Preservative | | | 0.3 | 0.3 | 0.3 | 0.3 | 0.3 | 0.3 |
| | | Active agent | 0.1 | | | | | |
| Detomidine HCl | | Active agent | | 1.0 | | | 1.0 | |
| | | Active agent | | | 2.0 | | | |
| | | Active agent | | | | 1.0 | | |
| | | Active agent | | | | | | 5.0 |
| Propane/butane | | Propellant | 10.0 | 10.0 | 10.0 | 1.0.0 | 1.0.0 | 10.0 |

In other embodiments, foam formulations containing detomidine HCl are prepared using the components and proportions listed in Table 20.

TABLE 20

| Component | % w/w | | | | |
|---|---|---|---|---|---|
| Mineral oil | 6.00 | 6.00 | 6.00 | 6.00 | 12.00 |
| Isopropyl myristate | 6.00 | 6.00 | 6.00 | 6.00 | 12.00 |
| Glyceryl monostearate | 0.50 | 0.50 | 0.50 | 0.50 | 1.00 |
| Stearyl alcohol | 1.00 | 1.00 | 1.00 | 1.00 | 1.00 |
| Xanthan Gum | 0.30 | 0.30 | 0.30 | 0.30 | 0.30 |
| Methocel K100M | 0.30 | 0.30 | 0.30 | 0.30 | 0.30 |
| TWEEN 60 | 1.00 | | | | |
| TWEEN 80 | | 1.00 | 1.00 | 1.00 | 2.00 |
| MYRJ 49p | 3.00 | 3.00 | 3.00 | 3.00 | 4.00 |
| Lactic acid | 10.00 | 5.00 | | | |
| Glycofurol | | | 1.00 | 10.00 | |
| Urea | | | | | 10.00 |
| Cocamidopropyl betaine | — | 0.50 | — | 0.50 | — |
| Detomidine HCl | 4.00 | 4.00 | 4.00 | 4.00 | 4.00 |
| Phenonip | 0.30 | 0.30 | 0.30 | 0.30 | 0.30 |
| Butane/propane | 8 | 8 | 6 | 10 | 10 |
| Water | to 100 | to 100 | to 100 | to 100 | to 100 |
| Foam Quality | E | E | E | E | E |
| Density | 0.028 | 0.038 | 0.044 | 0.028 | 0.028 |
| Mineral oil | 6.00 | 6.00 | 6.00 | 22.00 | 6.00 |
| Isopropyl myristate | 6.00 | 6.00 | 6.00 | 22.00 | 6.00 |
| Glyceryl monostearate | 0.50 | 0.50 | 0.50 | 1.00 | 0.50 |
| Stearyl alcohol | 1.00 | 1.00 | 1.00 | 1.00 | 1.00 |
| Xanthan gum | 0.30 | 0.30 | 0.30 | 0.40 | 0.30 |
| Methocel K100M | 0.30 | 0.30 | 0.30 | 0.40 | 0.30 |
| TWEEN 80 | | 1.00 | 1 .00 | 2.00 | 1.00 |
| MYRJ 49p | 1.00 | 3.00 | 3.00 | 4.00 | 3.00 |
| Propylene glycol | 3.00 | | | | |
| Transcutol p | | 10.00 | 1.00 | 2.00 | 10.00 |
| Cocamidopropyl | 0.50 | 0.50 | 0.50 | 0.50 | 0.50 |
| betaine | | | | | |
| Lidocaine base | 4.00 | 4.00 | 4.00 | 4.00 | 4.00 |
| Phenonip | 0.30 | 0.30 | 0.30 | 0.30 | 0.30 |
| Butane/propane | 8 | 8 | 6 | 8 | 10 |
| Water | to 100 | to 100 | to 100 | to 100 | to 100 |
| Foam Quality | E | E | E | E | E |
| Density | 0.032 | 0.035 | 0.048 | 0.075 | 0.025 |

In yet other embodiments, foam formulations containing detomidine HCl are prepared using the components and proportions listed in Table 21.

TABLE 21

| Comment | % w/w | | | |
|---|---|---|---|---|
| Mineral oil | 6.00 | 6.00 | 6.00 | 6.00 |
| Isopropyl myristate | 6.00 | 6.00 | 6.00 | 6.00 |
| Glyceryl monostearate | 0.50 | 0.50 | 0.50 | 0.50 |
| Stearyl alcohol | 0.20 | 0.20 | 0.20 | 1.00 |
| Detomidine HCl | 0.01-10 | 0.01-10 | 0.01-10 | 0.01-10 |
| Xanthan Gum | 0.30 | 0.30 | 0.30 | 0.30 |
| Methocel K100M | 0.30 | 0.30 | 0.30 | 0.30 |
| Myrj 52 | | | | 3.00 |
| TWEEN 80 | | | | 1.00 |
| MYRJ 49p | | | 3.00 | |
| TWEEN 60 | 1.00 | 1.00 | 1.00 | |
| Cocamidopropyl | 0.50 | 0.50 | 0.50 | |
| betaine | | | | |
| Phenonip | 0.30 | 0.30 | 0.30 | 0.30 |
| Butane/propane | 8.00 | 8.00 | 8.00 | 6.00 |
| Water | to 100 | to 100 | to 100 | to 100 |
| Foam Quality | E | E | E | E |
| Density | n/a | 0.023 | n/a | 0.044 |
| Mineral oil | 6.00 | | 6.00 | |
| Isopropyl myristate | 6.00 | | 6.00 | |
| Glyceryl monostearate | 0.50 | | 0.50 | |
| Stearyl alcohol | 1.00 | | 1.00 | |
| Urea | 40.00 | 40.00 | 20.00 | 20.00 |
| Xanthan gum | 0.30 | 0.30 | 0.30 | 0.30 |
| Methocel K100M | 0.30 | 0.30 | 0.30 | 0.30 |
| Myrj 52 | 3.00 | 3.00 | 3.00 | 3.00 |
| TWEEN 80 | 1.00 | 1.00 | 1.00 | 1.00 |
| Cocamidopropyl | 0.50 | 0.50 | | 0.50 |
| betaine | | | | |
| Phenonip | 0.30 | 0.30 | 0.30 | 0.30 |
| Butane/propane | 8.00 | 6.00 | 8.00 | 8.00 |
| Water | to 100 | to 100 | to 100 | to 100 |
| Foam Quality | E | E | E | E |
| Density | 0.022 | 0.029 | 0.032 | 0.024 |

### Example 14—Additional Spray Patch Formulations

Spray patch formulations containing detomidine HCl are prepared using components and proportions as described in Table 22.

TABLE 22

| | Formulation | | | | | | | |
|---|---|---|---|---|---|---|---|---|
| | 1 | 2 | 3 | 3A | 3B | 4 | 5 | 6 |
| Viscosity (cP) | 28,460 | 47,800 | 67,800 | 79,500 | 122,500 | 370,500 | 454,500 | 828,500 |
| Ingredients | | | | | | | | |
| Detomidine | 7.00% | 7.00% | 7.00% | 7.00% | 7.00% | 7.00% | 7.00% | 7.00% |
| Purified Water | 54.27% | 37.94% | 51.87% | 39.94% | 35.94% | 35.94% | 25.94% | 25.94% |
| DiCalcium | — | 24.00% | — | 18.00% | 27.00% | 27.00% | 36.00% | 36.00% |
| Phosphate | | | | | | | | |
| Polyvinyl Alcohol | 21.60% | 12.00% | 24.00% | 14.00% | 14.00% | 14.00% | 12.00% | 12.00% |
| White Petrolatum | 8.00% | 10.00% | 8.00% | 10.00% | 5.00% | 5.00% | 10.00% | 10.00% |
| Span 40 | 2.00% | 2.00% | 2.00% | — | — | 4.00% | 2.00% | 2.00% |
| (Sorbitan | | | | | | | | |
| Monopalmitate) | | | | | | | | |
| Span 60 | — | — | — | 4.00% | 4.00% | | | |
| (Sorbitan | | | | | | | | |
| Monostearate) | | | | | | | | |
| Methyl paraben | 0.10% | 0.05% | 0.10% | 0.05% | 0.05% | 0.05% | 0.05% | 0.05% |
| Propylparaben | 0.03% | 0.01% | 0.03% | 0.01% | 0.01% | 0.01% | 0.01% | 0.01% |
| Total | 100% | 100% | 100% | 100% | 100% | 100% | 100% | 100% |

In other embodiments, spray patch formulations containing detomidine HCl are prepared using the components and proportions listed in Table 23.

TABLE 23

| | Quantity range | Example 1 |
|---|---|---|
| DynamX ® | 0.1-40% (w/w) | 10% (w/w) |
| Triethyl Citrate | 0.1-18% (w/w) | 1% (w/w) |
| Water | 0.1-50% (w/w) | 16 (w/w) |
| Ethanol | 32-99.7% (w/w) | 68% (w/w) |
| Detomidine HCl | 0.1-30% (w/w) | 5% (w/w) |

In yet other embodiments, spray patch formulations containing detomidine HCl are prepared using the components and proportions listed in Table 24.

TABLE 24

| | Quantity | | | | | | |
|---|---|---|---|---|---|---|---|
| | Range | A | B | C | D | E | F |
| DynamX ® | 0.1-40% | 10% | 10% | 10% | 10% | 10% | 10% |
| Triethyl Citrate | 0.1-18% | 1% | 1% | 1% | 1% | 1% | 1% |
| Oleic Acid | 0.1-10% | 5% | 2.5% | 1.5% | | | |
| R-(+)-Limonene | 0.1-10% | | | | 5% | 2.5% | |
| IsopropylMyristate | 0.1-10% | | | | | | 5% |
| Propylene Glycol | 0.1-10% | | 2.5% | 1.5% | | 2.5% | |
| Ethanol | 22-99.8% | 62.2% | 62.2% | 62.2% | 62.2% | 62.2% | 62.2% |
| Water | 0.1-50% | 16.8% | 16.8% | 16.8% | 16.8% | 16.8% | 16.8% |
| Detomidine HCl | 0.1-30% | 5% | 5% | 5% | 5% | 5% | 5% |

%-Data = means % (w/w)

In additional embodiments, spray patch formulations containing detomidine HCl are prepared using the components and proportions listed in Table 25.

TABLE 25

| Formulation | | EUD | DYN | KLU | SIL | REF |
|---|---|---|---|---|---|---|
| Polymer | | Eudragit ® | DynamX ® | Klucel ® | SGM 36 | — |
| | | RL PO | | LF | | |
| Polymer | [%] | 20.0 | 10.0 | 5.0 | 10.0 | — |
| Content | | | | | | |
| Triethyl | [%] | 6.0 | 1.0 | 1.0 | — | — |
| Citrate | | | | | | |
| Ethanol | [%] | 69.0 | 67.2 | 89.0 | 25.0 | 95.0 |
| Water | [%] | — | 16.8 | — | — | — |
| Q7-9180 | [%] | — | — | — | 51.5 | — |
| 193 Fluid | [%] | — | — | — | 8.5 | — |
| Detomidine | [%] | 5.0 | 5.0 | 5.0 | 5.0 | 5.0 |

In yet other embodiments, spray patch formulations containing detomidine HCl are prepared using the components and proportions listed in Table 26.

TABLE 26

| | Formulation | Formulation | Formulation | Formulation |
|---|---|---|---|---|
| Components | 1 (%) | 2 (%) | 3 (%) | 4 (%) |
| PVP | 1.1 | 99.5 | 1.1 | — |
| Detomidine | 0.3 | — | 0.1 | — |
| EtOH | 10.5 | — | 10.6 | 9.3 |
| H₂O | 1.3 | 0.5 | 0.2 | 0.9 |
| HFA | 86.8 | — | 88.0 | 89.88 |
| Result | Insoluble | Soluble | Soluble | Soluble |

In yet other embodiments, spray patch formulations containing detomidine HCl are respectively prepared using the components and proportions listed in Tables 27-30.

TABLE 27
10% EtOH drug, HFA, PVP formulation composition

| Excipient | Formulation | Description |
|---|---|---|
| PVP | 2.46% | 2.7% |
| Detomidine | 0.09% | 0.1% |
| EtOH | 9.83% | — |
| HFA | 87.62% | 97.2% |

TABLE 28

| | Copovidone- | PVP | Eudragit | | | |
|---|---|---|---|---|---|---|
| Detomidine | S630 | K90 | RSPO | Ethanol | IPA | HFA |
| 0.0294 | — | 1.7995 | — | 8.7974 | — | 89.3737 |
| 0.0294 | 1.1247 | — | — | 4.9485 | — | 93.8974 |
| 0.0294 | — | 1.5183 | 1.5183 | 8.7974 | — | 88.1366 |
| 0.0294 | 1.3496 | — | — | — | 6.5981 | 92.0229 |

TABLE 29

| | PVP | Endragit | Copovidone- | | |
|---|---|---|---|---|---|
| Detomidine | K25 | RSPO | S-630 | Ethanol | HFA |
| 2.000 | — | 1.764 | — | 9.702 | 86.534 |
| 2.000 | — | — | 2.558 | 15.631 | 79.811 |
| 2.000 | 1.985 | — | — | 19.404 | 76.612 |
| 2.000 | 1.294 | 1.294 | — | 19.404 | 76.009 |

TABLE 30

| | PVP | Copovidone- | Endragit | | |
|---|---|---|---|---|---|
| Detomidine | K90 | S-630 | RSPO | Ethanol | HFA |
| 0.050 | 2.208 | — | — | 8.500 | 89.242 |
| 0.050 | — | 1.920 | — | 4.00 | 94.03 |
| 0.050 | 1.340 | — | 1.340 | 8.000 | 89.270 |

In still other embodiments, spray patch formulations containing detomidine HCl are prepared using the components and proportions listed in Table 31.

TABLE 31

| Ingredient | | | | | | | | | | | |
|---|---|---|---|---|---|---|---|---|---|---|---|
| (% w/v) | M0 | M1 | M2 | M3 | M4 | M5 | N1 | N2 | N3 | N4 | N5 |
| Detomidine | 1 | 1 | 1 | 1 | 1 | 1 | 1 | 1 | 1 | 1 | 1 |
| Eudragit E100 | 8 | 4 | 6 | 8 | 10 | 10 | 4 | 6 | 8 | 10 | 10 |
| Propylene glycol | 3 | 2 | 3 | 3 | 3 | 5 | — | — | — | — | — |
| Polyethylene | — | — | — | — | — | — | 3 | 5 | 5 | 5 | 3 |
| glycol 400 | | | | | | | | | | | |
| Camphor:Menthol | — | 10 | 10 | 10 | 10 | 10 | 10 | 10 | 10 | 10 | 10 |
| (1:1) | | | | | | | | | | | |
| Ethanol:Acetone | q.s. | q.s. | q.s. | q.s. | q.s. | q.s. | q.s. | q.s. | q.s. | q.s. | q.s. |
| (80:20) | | | | | | | | | | | |

In further embodiments, spray patch formulations containing detomidine HCl are respectively prepared using the components and proportions listed in Tables 32a and 32b.

TABLE 32a

| % ingredient w/w | B1 | B2 | B3 | B4 | B5 | B6 |
|---|---|---|---|---|---|---|
| Detomidine | 0.5 | 0.5 | 0.5 | 0.5 | 0.5 | 0.5 |
| Eudragit RLPO | 5 | 5 | 5 | 10 | 10 | 10 |
| EC (7 cPS) | 2.5 | 5 | 7.5 | 2.5 | 5 | 7.5 |
| Eutectic blend | 10 | 10 | 10 | 10 | 10 | 10 |
| PEG 400 | 0.45 | 0.45 | 0.45 | 0.45 | 0.45 | 0.45 |
| Alcohol and acetone | 100 | 100 | 100 | 100 | 100 | 100 |
| blend (q.s.) | | | | | | |

TABLE 32b

| % ingredient w/w | B7 | B8 | B9 | B10 | B11 |
|---|---|---|---|---|---|
| Detomidine | 0.5 | 0.5 | 0.5 | 0.5 | 0.5 |
| Eudragit RLPO | 15 | 15 | 15 | 10.0 | 10.05 |
| EC (7 cPs) | 2.5 | 5 | 7.5 | 5.0 | 5.02 |
| Eutectic blend | 10 | 10 | 10 | — | 10 |
| PEG 400 | 0.45 | 0.45 | 0.45 | 0.45 | 0.45 |
| Alcohol and | 100 | 100 | 100 | 100 | 100 |
| acetone blend (q.s.) | | | | | |

### Example 15—Additional Foam Formulations

Foam formulations of detomidine HCl were prepared by mixing the components and proportions as described in Table 33 in 20 ml scintillation vials and stirring by vortexing. Formulations were then transferred to an unpressurized vacuum pump and assessed for their foaming properties upon release from the pump.

TABLE 33

| | composition | | | | | | | | | |
|---|---|---|---|---|---|---|---|---|---|---|
| | A | B | C | D | E | F | G | H | I | J |
| | actual weight/g | | | | | | | | | |
| Benzalkonium | 1.5 | 1.5 | 1.5 | 1.5 | 1.5 | 1.5 | 1 | 1 | 1 | 1 |
| Chloride | | | | | | | | | | |
| Glyceryl | 1 | 1 | | | | | | | | |
| Isostearate | | | | | | | | | | |
| Triacetin | 1 | 1 | 2 | 2 | 5 | 5 | | | | |
| Dowsil ™ | | | 3 | 3 | | | | | | |
| 5225C | | | | | | | | | | |
| Formulation | | | | | | | | | | |
| Aid | | | | | | | | | | |
| Triethylcitrate | | | | | | | 4 | 4 | 6 | 6 |
| Paraffin | 2 | 2 | | | | | | | | |
| (highly liquid) | | | | | | | | | | |
| Detomidine | 4.1 | 0.01 | 3.5 | 0.01 | 3.4 | 0.01 | 2.2 | 0.01 | 2.4 | 0.01 |
| 8.0% in | | | | | | | | | | |
| double | | | | | | | | | | |
| distilled water | | | | | | | | | | |
| Double | 8.4 | 12.5 | 7.2 | 10.7 | 7 | 10.4 | 4.8 | 7 | 5.0 | 7.4 |
| distilled water | | | | | | | | | | |

All foam compositions were satisfactory to good in quality with compositions A-C demonstrating the best properties. Surprisingly, it was also identified that compositions of 3% detomidine produced better foams than similar compositions containing only 0.01%.

1. A topical formulation comprising:
about 0.001 to 3% by weight of detomidine or a salt thereof; and,
a carrier that is suitable for topical administration to a human subject's skin,
wherein the carrier comprises a hydrophilic phase member and a hydrophobic phase member in a ratio of not more than 49.9%, and,
wherein the detomidine is fully dissolved and chemically stable in the hydrophilic phase member.

2. The topical formulation according to claim 1, wherein the concentration of detomidine dissolved in the hydrophilic phase member is up to 40% w/w, is about 0.04-35% w/w, or is about 10-30% w/w.

3. (canceled)

4. (canceled)

5. The topical formulation according to claim 1, wherein the formulation is prepared by (i) dissolving the detomidine or salt thereof in the hydrophilic phase member, and (ii) combining the hydrophilic phase member containing the dissolved detomidine or salt thereof with the hydrophobic phase member in order to form the topical formulation.

6. The topical formulation according to claim 1, comprising about 0.005 to 2% by weight of the detomidine or salt thereof, about 0.01 to 2% by weight of the detomidine or salt thereof, about 0.05 to 1% by weight of the detomidine or salt thereof, about 0.08 to 0.5% by weight of the detomidine or salt thereof, about 0.08 to 0.2% by weight of the detomidine or salt thereof, or about 0.1% by weight of the detomidine or salt thereof.

7. (canceled)

8. (canceled)

9. (canceled)

10. (canceled)

11. (canceled)

12. The topical formulation according to claim 1, wherein the hydrophilic phase member is water, glycerol, polypropylene glycol, polyethylene glycol, ethanol, benzyl alcohol, propylene carbonate, 2-(2-ethoxyethoxy)ethanol, dimethyl isosorbide, tetraglycol, pyrrolidone, dimethylacetamide, caprylocaproyl polyoxyl-8 glycerides, or any combination thereof.

13. The topical formulation according to claim 1, wherein the hydrophilic phase member includes an aqueous buffer solution.

14. The topical formulation according to claim 13, wherein the hydrophilic phase member comprises 0.01 to 1.0M citrate, phosphate, Tris, carbonate, succinate, tartrate, borate, imidazole, maleate, or phthalate buffer at pH 4.5-9.0.

15. The topical formulation according to claim 13, wherein the hydrophilic phase member comprises 0.1M citrate buffer at pH 6.1, 0.1M phosphate buffer at pH 6.2, 0.1M phosphate buffer at pH 7.2, 0.1M Tris buffer at pH 8.2, 0.4M Tris buffer at pH 8.2, or water with 60% w/w PEG 3350 in pH 6.2 buffer.

16. The topical formulation according to claim 1, wherein the hydrophobic phase member is an aromatic hydrocarbon, an alkane, a cycloalkane, an alkyne, a terpene, an organic oil, a mineral oil, or any combination thereof.

17. The topical formulation according to claim 1, wherein the hydrophilic phase member and a hydrophobic phase member are present in the carrier in a ratio of less than about 20%, less than about 10%, or less than about 5%.

18. (canceled)

19. (canceled)

20. The topical formulation according to claim 1, further comprising one or more of a thickening agent, a preservative, a permeation enhancer, a wax, an emulsifying agent, an emollient, a humectant, a conditioning agent, antioxidant, and a viscosity regulator.

21. The topical formulation according to claim 1, further comprising a further therapeutic agent in addition to the detomidine or salt thereof.

22. The topical formulation according to claim 21, wherein said further therapeutic agent comprises an analgesic.

23. The topical formulation according to claim 1, comprising a foam.

24. The topical formulation according to claim 23, wherein the hydrophobic phase member comprises a medium chain triglyceride.

25. The topical formulation according to claim 23, wherein the hydrophilic phase member comprises one or more of propylene glycol, hexylene glycol, or water.

26. The topical formulation according to claim 23, wherein the hydrophobic phase member comprises a mineral oil.

27. The topical formulation according to claim 23, wherein the hydrophilic phase member comprises water.

28. The topical formulation according to claim 1, comprising a cream.

29. The topical formulation according to claim 28, wherein the hydrophobic phase member comprises one or more of mineral oil, isopropyl isostearate, isostearyl isostearate, alkyl benzoate, butyl stearate, diisopropyl adipate, diethylhexyl adipate, caprilic/capric triglyceride, isocetyl stearate, isopropyl myristate, isopropyl palmitate, lauryl lactate, myristil myristate, ethylhexyl cocoate, ethylhexyl palmiatate, ethylhexyl pelagronate, ethylhexyl stearate, diethylhexyl succinate, propylene glycol dicaprylate/dicaprate, PPG-2 myristyl ether propionate, pentaerythrityl tetracaprylate/caprate, pentaerythrityl tetraisostearate, or isotridecyl isononanoate.

30. The topical formulation according to claim 28, wherein the hydrophilic phase member comprises one or more of glycerol, propylene glycol, water, 1,3-propanediol, 1,2-pentanediol, hexylene glycol, or butylene glycol.

31. The topical formulation according to claim 28, comprising a fatty alcohol, an ester of a fatty alcohol, or both, an emulsifier, and an emollient.

32. The topical formulation according to claim 1, further comprising porous microparticles or nanoparticles, at least some of the pores of said microparticles or nanoparticles being loaded with detomidine, a further active pharmaceutical agent, or both.

33. The topical formulation according to claim 32 wherein the microparticles or nanoparticles have an average pore diameter of about 0.05 to 0.5 μm.

34. The topical formulation according to claim 32, wherein the microparticles or nanoparticles comprise poly(lactic-co-glycolic acid), poly(lactic acid), poly(l-lactic) acid, chitosan, methyl methacrylate/ethylene glycol dimethacrylate crosspolymer, a cellulose ether polymer, or a methacrylate polymer.

35. The topical formulation according to claim 1, comprising a microemulsion gel.

36. The topical formulation according to claim 35, wherein the microemulsion gel comprises up to 10% water, up to 20% by weight of a surfactant, a co-surfactant, or both, and 80-100% by weight of an oil.

37. The topical formulation according to claim 35, wherein the microemulsion gel comprises up to 10% by weight water, 30-40% by weight of a surfactant, a co-surfactant, or both, and 60-70% by weight of an oil.

38. The topical formulation according to claim 35, comprising a silicone-based emulsifier or gelling agent.

39. The topical formulation according to claim 38, wherein the silicone-based emulsifier is liquid alkylmethyl silicone polyether copolymer, ethoxylated and propoxylated silicone surfactant, cyclopentasiloxane, or cyclopentasiloxane with polyethylene glycol/polypropylene glycol-19/19 dimethicone.

40. The topical formulation according to claim 1, comprising an oil-in-water emulsion.

41. The topical formulation according to claim 40, wherein the hydrophobic phase member comprises PEG 2 stearyl ether, steareth-21, or both.

42. An topical formulation comprising:
about 0.001 to 10% by weight of detomidine or a salt thereof; and,
a carrier that is suitable for topical administration to a human subject's skin,
wherein the carrier comprises a hydrophobic phase member and a hydrophilic phase member in a ratio of not more than 49.9%; and,
wherein the detomidine is fully dissolved and chemically stable in the hydrophilic phase member.

43. The topical formulation according to claim 42, wherein the carrier comprises a stiffening agent, an emulsifier, and an emollient.

44. The topical formulation according to claim 43, wherein stiffening agent is a fatty alcohol, an ester thereof, or both.

45. The topical formulation according to claim 43, comprising a cream.

46. The topical formulation according to claim 42, comprising an oil-in-water emulsion in which porous microparticles or nanoparticles are dispersed, at least some of the pores of said microparticles or nanoparticles being loaded with detomidine or a salt thereof, a further active pharmaceutical agent, or both.

47. The formulation according to claim 46, wherein the microparticles or nanoparticles have an average pore diameter of about 0.05 to 0.5 μm.

48. The topical formulation according to claim 46, wherein the microparticles or nanoparticles comprise poly(lactic-co-glycolic acid), poly(lactic acid), poly(l-lactic) acid, chitosan, methyl methacrylate/ethylene glycol dimethacrylate crosspolymer, a cellulose ether polymer, or a methacrylate polymer.

49. The topical formulation according to claim 42, comprising an emulgel.

50. The topical formulation according to claim 42, comprising a micro-emulsion gel.

51. The topical formulation according to claim 50, comprising a Polyamide-3, Polyamide-4, polyvinyl pyrrolidone, or polyvinyl alcohol gelling agent.

52. The topical formulation according to claim 50, comprising a silicone-based emulsifier or gelling agent.

53. The topical formulation according to claim 52, wherein the silicone-based emulsifier is liquid alkylmethyl silicone polyether copolymer, ethoxylated and propoxylated silicone surfactant, cyclopentasiloxane, or cyclopentasiloxane with polyethylene glycol/polypropylene glycol-19/19 dimethicone.

54. A topical formulation comprising an oil-in-water emulsion in which porous microparticles or nanoparticles are dispersed, at least some of the pores of said microparticles or nanoparticles being loaded with detomidine or a salt thereof, a further active pharmaceutical agent, or both.

55. The topical formulation according to claim 54, wherein the microparticles or nanoparticles have an average pore diameter of about 0.05 to 0.5 μm.

56. The topical formulation according to claim 54, wherein the microparticles or nanoparticles comprise poly(lactic-co-glycolic acid), poly(lactic acid), poly(l-lactic) acid, chitosan, methyl methacrylate/ethylene glycol dimethacrylate crosspolymer, a cellulose ether polymer, or a methacrylate polymer.

57. A sprayable composition comprising detomidine or a salt thereof that forms a transdermal patch when sprayed onto the subject's skin.

58. A topical liposomal composition for delivering a drug comprising:
lipid components between 5% to 30% w/w, wherein said lipid components comprise phospholipid and cholesterol;
a solvent; and,
a plurality of additives selected from the group consisting of penetration enhancers, emulsifiers, antioxidants, buffering agents, pH adjusting agents, antimicrobial preservatives, one or more gelling agents, thickening agent, emollient, skin conditioner, viscosity increasing agent, film-forming agent, absorbent, water, and any combination thereof,
wherein said the drug is detomidine or a salt thereof.

59. The topical liposomal composition according to claim 58, wherein the amount of detomidine or salt thereof is about 0.1% to about 1% by weight based on the total weight of the composition.

60. The topical liposomal composition according to claim 58, wherein said phospholipid is selected from a group consisting of phosphatidylcholine (PC) distearoyl phosphatidylcholine (DSPC), dipalmitoyl phosphatidylcholine (DPPC), dimirystoyitoyl phosphatidylcholine (DMPC), dilauroyl phosphatidylcholine (DLPC), soya phosphatidylcholine (SPC), egg phosphatidylcholine (EPC), hydrogenated soya phosphatidylcholine (HSPC), hydrogenated egg phosphatidylcholine (HEPC), and combinations thereof.

61. The topical liposomal composition according to claim 58, wherein liposomes have an average diameter less than about 150 nm.

62. The topical liposomal composition according to claim 58, wherein said plurality of additives comprise a penetration enhancer selected from the group consisting of oleic acid, propylene glycol, and a combination thereof.

63. The topical liposomal composition according to claim 58, wherein said plurality of additives comprise an antimicrobial preservative selected from the group consisting of methyl paraben (MP), propyl paraben (PP), phenol, and combinations thereof.

64. The topical liposomal composition according to claim 58, wherein said solvent comprises at least two solvents for dissolving said lipid components, and wherein said at least the two solvents are propylene glycol and glycerol in a combined amount of about 1% to about 25% by weight based on the total weight of the composition.

65. The topical liposomal composition according to claim 58, wherein the composition is formulated in the form of a cream, a serum, a lotion, or a gel.

66. A method for preparing a topical liposomal composition comprising:
dissolving lipid components in a solvent;
mixing the dissolved lipid components in said solvent with a plurality of additives selected from the group consisting of a penetration enhancer, an antimicrobial preservative, an antioxidant, and combinations thereof, thereby forming a lipid phase thereof;
melting said lipid phase in order to form a uniform dissolved lipid phase;
dissolving and mixing one hydrophilic drug into the solvent in the uniform lipid phase, thereby forming an admixture thereof;
heating said admixture;
dissolving an emulsifier into a solution, thereby preparing an aqueous phase thereof;
heating said solution in said aqueous phase; and,
mixing and homogenizing said admixture and said solution in said aqueous phase, thereby producing said topical liposomal composition.

67. The method according to claim 66, further comprising adding one or more gelling agent, thickening agent, emollient, skin conditioner, viscosity increasing agent, film-forming agent, or absorbent to the topical liposomal composition.

68. A method for providing prolonged, non-systemic treatment for pain in a human subject in need thereof comprising topically administering to the human subject a topical formulation according to claim 1.

69. The method according to claim 68, wherein said pain is neuropathic pain, erythromelalgia, diabetic neuropathic pain, or postherpetic neuralgia.

70. (canceled)

71. (canceled)

72. (canceled)

73. The method according to claim 68, wherein the topical formulation is topically administered to the human subject once- or twice-daily basis.

74. A method for providing treatment for rosacea in a human subject in need thereof comprising topically administering to the human subject a topical formulation according to claim 1.

75. The method according to claim 74, wherein the topical administration is to areas of the subject's skin that include rosacea discoloration.
